(19) United States
(12) Patent Application Publication
(10) Pub. No.: US 2025/0114478 A1
(43) Pub. Date: Apr. 10, 2025

(54) LIPID NANOPARTICLE (LNP) COMPOSITIONS FOR BRAIN-SELECTIVE CARGO DELIVERY, AND METHODS OF USE THEREOF

(71) Applicants: THE TRUSTEES OF THE UNIVERSITY OF PENNSYLVANIA, Philadelphia, PA (US); THE CHILDREN'S HOSPITAL OF PHILADELPHIA, Philadelphia, PA (US)

(72) Inventors: Michael J. Mitchell, Philadelphia, PA (US); Rohan Palanki, Philadelphia, PA (US); William Peranteau, Philadelphia, PA (US)

(21) Appl. No.: 18/907,152

(22) Filed: Oct. 4, 2024

Related U.S. Application Data
(60) Provisional application No. 63/588,493, filed on Oct. 6, 2023.

(51) Int. Cl. A61K 48/00; A61K 9/51; C12N 15/113

(52) U.S. Cl. CPC A61K 48/0033; A61K 9/5123; C12N 15/113; C12N 2310/11; C12N 2310/113; C12N 2310/14; C12N 2310/141

(57) ABSTRACT

In one aspect, the present disclosure relates to lipid nanoparticles (LNPs) comprising at least one ionizable lipid, at least one helper lipid, cholesterol, at least one conjugated lipid, and at least one nucleic acid cargo (e.g., at least one mRNA and at least one sgRNA). In another aspect, the present disclosure relates to a method of delivering a cargo to the brain of a subject in need thereof. In another aspect, the present disclosure relates to methods of genome editing a mutated gene sequence associated with a disease or disorder in a subject in need thereof. In another aspect, the present disclosure relates to methods of treating a lysosomal storage disease in a subject in need thereof. In certain embodiments, the LNPs of the present disclosure are administrated in utero and/or intracerebroventricularly. In certain embodiments, the subject is embryonic, fetal, neonatal, or perinatal.

### CROSS-REFERENCE TO RELATED APPLICATIONS

This application claims priority under 35 U.S.C. § 119 (e) to U.S. Provisional Patent Application No. 63/588,493, filed Oct. 6, 2023, which is incorporated herein by reference in its entirety.

### STATEMENT REGARDING FEDERALLY SPONSORED RESEARCH OR DEVELOPMENT

This invention was made with government support under TR⁰⁰²⁷⁷⁶ and HL152427 awarded by the National Institutes of Health. The government has certain rights in the invention.

### SEQUENCE LISTING

The XML file named “046483-7398US1_Sequence_Listing.xml” created on Sep. 25, 2024, comprising 14.4 Kbytes, is hereby incorporated by reference in its entirety.

### BACKGROUND

Approximately 7,000 to 10,000 diseases can be attributed to mutations in single genes, of which 17% have a significant neurologic component (e.g., Friedrich's ataxia, Huntington's disease) and 10% are inborn errors of metabolism (e.g., lysosomal storage diseases, mitochondrial disorders) that often have severe neurologic symptoms. Together, these congenital diseases affect approximately 1% of children at birth and are associated with a wide array of pathologic central nervous system (CNS) features, including developmental delay and progressive neuronal degeneration, structural disruption (e.g., microcircuit impairment, brain atrophy), and motor and cognitive dysfunction. Although some genetic and metabolic diseases affecting the CNS can be treated with enzyme replacement therapies, few curative therapies exist and current clinical management for most genetic CNS disorders focuses primarily on symptom reduction. As a result, these diseases have significant morbidity and mortality, accounting for 40% of pediatric hospitalizations worldwide.

Advances in gene editing technology provide a unique opportunity for a “one- and -done” treatment for monogenic diseases. Clustered regularly interspaced short palindromic repeat (CRISPR) and CRISPR-associated protein 9 (Cas9) platforms, which induce a double-stranded DNA break (DSB) at a specific location based on a single guide RNA (sgRNA) target sequence to stimulate endogenous repair mechanisms, are in early stage clinical trials for a number of diseases including in vivo therapeutic gene editing trials for Leber's congenital amaurosis, hereditary angioedema, and transthyretin amyloidosis.

The toolbox for gene editing has expanded in recent years with the introduction of DNA base editors, which comprise a catalytically inactive Cas9 fused to an adenine or cytosine deaminase. Adenine (ABE) and cytosine base editors (CBE) can effect purine-to-purine and pyrimidine-to-pyrimidine changes in a site specific manner without DSBs and the requirement for proliferating cells to function efficiently. Since single point mutations contribute to the majority of all known pathogenic mutations, base editors hold tremendous promise for the treatment of genetic disorders, including congenital brain diseases. Furthermore, the lack of a requirement for proliferating cells and a DSB, supports the potential of base editing to be more precise, efficient and safe than CRISPR/Cas9-mediated nonhomologous end-joining and homology directed repair.

The pathogenesis of many congenital CNS diseases begins in utero and is often irreversible. In recent years, prenatal detection of many structural and genetic diseases has become standard-of-care with the advent of high-resolution fetal ultrasonography, ultrafast fetal MRI, and advances in genetic sequencing including high sensitivity genetic analysis of cell-free fetal DNA present in maternal serum. These technologies have unlocked the potential to prenatally diagnose and subsequently treat disease before birth and the onset of irreversible pathology.

In addition to treating a genetic disease before a pathologic insult, in utero gene editing takes advantage of normal fetal ontogeny to deliver therapies in a potentially more efficient manner. Specifically, the small fetal size maximizes the dose of treatment per weight, accessible and abundant stem/progenitor cell populations in the fetus support the persistence of the therapeutic edit, and a tolerogenic fetal immune system minimizes an immune barrier to gene editing tools.

The potential of in utero gene editing has been previously demonstrated in mouse models of human diseases, including rescuing the lethal phenotype of hereditary tyrosinemia type 1, improving the pulmonary phenotype of surfactant protein C deficiency, and ameliorating metabolic, musculoskeletal, and cardiac disease in mucopolysaccharidosis type I (MPS-IH, Hurler syndrome). However, these studies involved the viral vector delivery of Streptococcus pyogenes Cas9 (SpCas9) or base editors and demonstrated efficient editing of the liver, heart and lung.

A key challenge for translation of messenger RNA (mRNA)-based gene therapies, including gene editing, is safe and effective intracellular delivery. Large size, anionic charge, and susceptibility to RNAses all hinder therapeutic mRNA from efficiently entering cells on their own. Owing to their intrinsic ability to transduce cells, viral delivery platforms (e.g., adenoviruses and adeno-associated viruses) have been utilized in most gene therapy clinical trials currently in progress. However, the efficacy of viral gene therapies can be limited by pre-existing viral immunity, viral-induced immunogenicity, payload size constraints, undesired vector integration, and adverse clinical events related to the viral vector.

To overcome some of these limitations, novel non-viral delivery technologies have been developed. Ionizable lipid nanoparticles (LNPs) are among the most promising non-viral delivery platforms, owing to their biocompatibility and efficacy in both pre-clinical and clinical models. LNPs have been widely used as delivery platforms for the Moderna and Pfizer SARS-CoV-2 mRNA vaccines and FDA-approved siRNA orphan drugs, such as Onpattro®, Givlaari®, and Oxlumo®. LNPs have also been used to deliver CRISPR-based gene editing therapies to the liver in postnatal mouse and non-human primate studies. Recently, LNPs were utilized in clinical trials to deliver ABE8.8 mRNA and sgRNA to inactivate the PCSK9 gene as a treatment for familial hypercholesterolemia and Cas9 mRNA and sgRNA to inactivate the TTR gene as a treatment for transthyretin amyloidosis.

For both approaches, the target organ was the liver, and early results from the transthyretin amyloidosis trial are encouraging, while the trial for familial hypercholesterolemia has just begun. A distinct advantage of using LNPs is their modularity, which makes them broadly applicable as delivery carriers, but this necessitates designing LNPs specifically for both the target delivery location, intended cargo and intended patient population. Previous work has led to the identification of LNPs for mRNA delivery to the fetal mouse liver following in utero intravascular injection. However, LNPs have not been studied as a therapeutic delivery vehicle in the perinatal brain.

There is thus a need in the art for LNP compositions suitable for delivery of nucleic acid cargo to the brain, and methods of use thereof for in utero gene editing. The present disclosure addresses this need.

### BRIEF SUMMARY

In one aspect, the disclosure provides a lipid nanoparticle (LNP) composition. In certain embodiments, the LNP composition comprises at least one ionizable lipid. In certain embodiments, the LNP composition comprises at least one helper lipid. In certain embodiments, the LNP composition comprises cholesterol. In certain embodiments, the LNP composition comprises at least one conjugated lipid. In certain embodiments, the LNP composition comprises nucleic acid cargo comprising at least one mRNA and at least one sgRNA, wherein the nucleic acid cargo are at least partially encapsulated in the LNP.

In certain embodiments, the at least one ionizable lipid comprises an ionizable lipid of Formula (I), or a salt, solvate, stereoisomer, or isotopologue thereof, wherein R^{1a}, R^{1b}, R^{2a}, R^{2b}, R^{2c}, R^{2d}, R^{2e}, R^{2f}, R^{2g}, and R^{2h} are defined elsewhere herein:

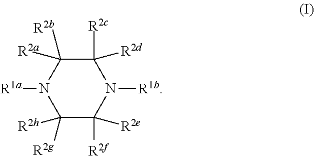

In certain embodiments, the ionizable lipid of Formula (I) is:

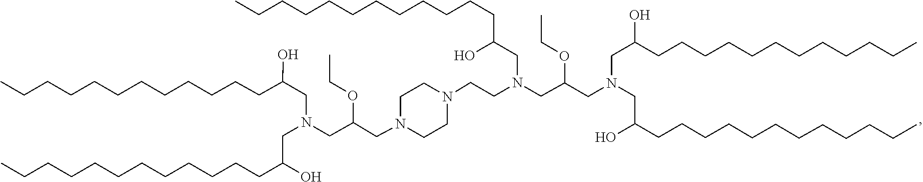 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]

### 1,1′-((3-(4-(2-((3-(bis(2-hydroxytetradecyl)amino)-2-ethoxypropyl) (2-hydroxytetradecyl)amino)ethyl) piperazin-1-yl)-2-ethoxypropyl) azanediyl)bis(tetradecan-2-ol) (C3)

In another aspect, the disclosure provides a pharmaceutical composition comprising the LNP of the disclosure and a pharmaceutically acceptable carrier.

In another aspect, the disclosure provides a method of delivering a cargo to the brain of a subject in need thereof. In certain embodiments, the method comprises administering to the subject a therapeutically effective amount of the LNP of the disclosure or a pharmaceutical composition thereof.

In another aspect, the disclosure provides a method of genome editing a mutated gene sequence associated with a disease or disorder in a subject in need thereof. In certain embodiments, the method comprises administering to the subject a therapeutically effective amount of the LNP of the disclosure or a pharmaceutical composition thereof.

In another aspect, the disclosure provides a method of treating ameliorating, and/or preventing a lysosomal storage disease in a subject in need thereof. In certain embodiments, the method comprises administering to the subject a therapeutically effective amount of the LNP of the disclosure or a pharmaceutical composition thereof.

### BRIEF DESCRIPTION OF THE FIGURES

The drawings illustrate generally, by way of example, but not by way of limitation, various embodiments of the present application.

FIG. 1 depicts engineering of LNPs for base editing in the perinatal brain. Chemical structures of epoxide-terminated alkyl tails and polyamine cores that were combined to generate the ionizable lipid library utilized herein are provided. Formulation of LNPs via microfluidic mixing with an ethanol phase containing ionizable lipid, PEG-lipid, cholesterol, and DOPE and an aqueous phase containing luciferase mRNA is also visualized. As described herein, LNPs were named based on the alkyl tail length (i.e., A=C12, B═C14, C═C16) and polyamine (PA) cores (i.e., 1 to 4) of the ionizable lipid incorporated into the formulation. After screening the LNP library via fetal and neonatal intracerebroventricular (ICV) injection followed by in vivo imaging system (IVIS) analysis, the top performing LNP was optimized in vitro for base editing applications prior to demonstration of LNP safety and efficacy in a neonatal murine disease model, fetal non-human primate (NHP), and patient-derived fluids and tissues.

FIGS. 2A-2F depict evaluation of a LNP library for perinatal brain mRNA delivery. FIG. 2A provides a graphical representation of the size and PDI of each LNP formulation in the LNP library. FIG. 2B provides additional characterization of the LNP library showing zeta potential (radius of circle), pKₐ (gradient), and encapsulation efficiency (centered number) for each LNP formulation. FIG. 2C depicts a schematic of an experimental design wherein LNPs were injected into individual E18 BALB/c fetuses or P0 BALB/c neonates via ICV injection (dose=1 mg/kg total mRNA). After 4 hours, animals were sacrificed, and tissues were extracted for IVIS imaging. FIG. 2D depicts IVIS imaging showing luciferase expression in a representative C3 LNP-treated fetus (left) and neonate (right) relative to PBS-treated controls. FIG. 2E depicts quantification of luciferase signal from the brains of fetuses treated with each LNP. FIG. 2F depicts quantification of luciferase signal from the brains of neonates treated with a subset of LNPs. All luminescence readings are represented as normalized total flux (luminescence of area of interest divided by background luminescence). ** p<0.01, **** p<0.0001 by one-way analysis of variance (ANOVA) with post hoc Dunnett's test compared to positive control (fetus-MC3, neonate-C3). Outliers were detected using Grubbs' test and removed from analysis; minimum n=3 per treatment group; error bars represent SEM.

FIGS. 3A-3B provide bar graphs depicting luciferase expression (FIG. 3A) and viability (FIG. 3B) of Neuro-2A cells after treatment with either C3 or MC3 LNP (100 ng of mRNA) encapsulating luciferase mRNA. ** p<0.01, **** p<0.001 by two-way analysis of variance (ANOVA) with post-hoc Šídák's multiple comparisons test.

FIGS. 4A-4C provide graphs showing the relationship between certain physiochemical properties, including pKₐ (FIG. 4A), size (FIG. 4B), and zeta potential (FIG. 4C) and LNP transfection efficacy in vivo after ICV injection to E18 Balb/c fetuses as measured by quantification of luciferase signal 4 hours after administration via IVIS imaging. Correlation coefficient (R) of linear regression analysis is reported in the top left corner of each individual graph.

FIG. 5 depicts quantification of luciferase signal from the livers of BALB/c fetuses treated with each LNP in the screening library.

FIG. 6 provides a bar graph depicting quantification of luciferase signal via IVIS imaging from harvested organs (i.e., brain, heart, lung, intestine, liver, spleen, and kidney) of E18 BALB/c fetuses 4 hours after ICV injection (1 mg/kg) with C3 LNP or PBS following normalization to background autofluorescence. **** p<0.001 by two-way analysis of variance (ANOVA) with post-hoc Šídák's multiple comparisons test.

FIGS. 7A-7E depict cellular tropism of C3 LNPs in the neonatal mouse brain. FIG. 7A depicts a schematic of an experimental design wherein C3 LNPs encapsulating Cre mRNA were injected into P0 R^{26mT/mG} neonates (n=4) via ICV injection (dose=1 mg/kg total mRNA). Animals were sacrificed one week later, and brain tissue was harvested for flow cytometry and immunohistochemistry analyses. FIG. 7B provides a graphical representation of the percentage of green fluorescent protein (GFP) positive cells among a heterogenous population of brain cells and sub-populations of microglia (CD11b⁺/CD45^{lo}), astrocytes (GFAP⁺/CD45⁻), and neurons (NFM⁺/CD45⁻). Error bars represent SEM. FIG. 7C depicts whole brain histology of neonatal brains 7 days after PBS, C1, C2, or C3 LNP ICV injection, displaying unedited (tdTomato+) and edited (GFP+) regions, imaged at 1×. FIG. 7D depicts representative histology focused on the brain ventricular lining adjacent to the site of C3 LNP ICV injection, imaged at 4× and 20×, displaying unedited (tdTomato+) and edited (GFP+) regions. FIG. 7E depicts representative histology focused on the brain ventricular lining with intracellular staining to capture successful LNP-mediated delivery (GFP+) to astrocytes (GFAP+), microglia (IBA1+), and neurons (NeuN+). Scale bars: 200 μm (4×) and 50 μm (20×).

FIGS. 8A-8G depict in vitro optimization of C3 LNPs for co-delivery of base editing platforms. FIG. 8A provides a schematic demonstrating the three LNP formulation parameters evaluated:excipient molar ratios, N:P ratio, and ABE to sgRNA mass ratio. FIG. 8B depicts an orthogonal design of experiments (DOE) array displaying excipient molar ratios used in C3 LNP library formulation. FIG. 8C depicts mean fluorescence intensity in Neuro-2a cells 24 hours after treatment of each formulation in the C3 LNP library (dose=100 ng total mRNA). Values were normalized to the original C3 LNP formulation, represented graphically by the dotted line. FIG. 8D depicts mean fluorescence intensity in Neuro-2a cells 24 hours after treatment with each C3 LNP formulation prepared at a range of N:P ratios (dose=100 ng total mRNA). Values were normalized to the original C3 LNP formulation (prepared at a 10:1 N:P ratio). FIG. 8E depicts quantification of luciferase signal from brains of neonates treated with C3 LNPs prepared at the top-performing N:P ratios. Values are reported as normalized total flux, as described previously. FIG. 8F provides a graphical representation of sequencing results at the expected site of base editing following C3 LNP-mediated base editing in primary MPS-I murine fibroblasts at three ABE/sgRNA mass ratio (dose=200 ng total mRNA). FIG. 8G provides a graphical representation of sequencing results in primary MPS-I murine neurons treated with C3.MPS LNPs at a range of doses. * p<0.05, ** p<0.01, *** p<0.001 by one-way ANOVA with post hoc Dunnett's test compared to each positive control as described in the text. Outliers were detected using Grubbs' test and removed from analysis; minimum n=3 replicates per treatment group; error bars represent SEM.

FIGS. 9A-9C depict impact trend curves of C3 ionizable lipid (FIG. 9A), DOPE (FIG. 9B), and cholesterol (FIG. 9C) on in vitro mRNA delivery efficiency.

FIGS. 10A-10B provide bar graphs depicting viability of Neuro-2a cells treated with each C3 LNP formulation prepared at different excipient ratios (FIG. 10A) or different N:P ratios in Neuro-2A cells. **** p<0.0001 by one-way analysis of variance (ANOVA) with post hoc Dunnett's test compared to control.

FIG. 11A provides a table depicting characterization data for C3.MPS LNPs. FIG. 11B depicts sequencing results at the expected site of base editing following C3 LNP-mediated base editing in primary MPS-I murine fibroblasts. FIG. 11C provides a bar graph depicting viability of primary MPS-I murine fibroblasts and primary MPS-I murine neurons, isolated from an IDUA-W392X mouse, after treatment with C3.MPS LNPs (200 ng of total mRNA).

FIGS. 12A-12E depict the genomic and biochemical efficacy of C3.MPS LNPs in Idua-W392X neonates. FIG. 12A provides a scheme for genetic, biochemical, and safety analysis of Idua-W392X neonates injected ICV at P0 with C3.MPS LNPs (dose=1 mg/kg total mRNA) and harvested one month after treatment. FIG. 12B depicts next-generation sequencing (NGS) results at the expected site of base editing in three different sections of harvested brain tissue. * p<0.05, by one-way analysis of variance (ANOVA) with post hoc Dunnett's test compared to control. FIG. 12C depicts iduronidase (IDUA) on-target activity measured after harvest in three different sections of brain tissue from B6 mice (positive control), C3.MPS LNP-treated Idua-W392X mice (experimental group), and untreated Idua-W392X mice (negative control), and top computationally predicted off-target sites (SEQ ID NOs: 1 and 7-15). FIG. 12D depicts NGS at the Idua on-target site and the top computationally predicted off-target sites in brain genomic DNA of two C3.MPS LNP-treated Idua-W392X mice and one negative control. FIG. 12E depicts glycosaminoglycans (GAGs) in three sections of brain tissue from B6, C3.MPS LNP-treated Idua-W392X, and untreated Idua-W392X mice. ** p<0.01, *** p<0.001, **** p<0.0001 by two-way analysis of variance (ANOVA) with post hoc Šídák's multiple comparisons test. Outliers were detected using Grubbs' test and removed from analysis; minimum n=4 per treatment group; mean represented by horizontal line for each group.

FIG. 13 depicts NGS results in control or C3.MPS LNP treated Idua-W392X neonates at the expected site of base editing in harvested liver and gonad tissue. No significant difference relative to control in either tissue as evaluated by Student's t test with a=0.05.

FIGS. 14A-14C depict the immune profile of C3.MPS LNPs in Idua-W392X neonates. FIG. 14A provides a bar graph depicting cytokine analysis in serum of Idua-W392X neonates treated 24 hours prior with C3.MPS LNPs. **** p<0.0001 by two-way analysis of variance (ANOVA) with post hoc Šídák's multiple comparisons test. FIG. 14B provides a bar graph depicting serum anti-PEG IgM antibody levels one week following C3.MPS LNP treatment. *** p<0.001 by Student's t test with a=0.05. Outliers were detected using Grubbs' test and removed from analysis; minimum n=3 per treatment group; error bars represent SEM. FIG. 14C provides images depicting hematoxylin and eosin (H&E) stained whole brain tissue sections of PBS- or C3.MPS-treated Balb/c neonates with focus on the lateral ventricle; scale bars: 1 mm (1×) and 50 μm (20×).

FIGS. 15A-15K depict C3 LNP-mediated in utero mRNA delivery to the NHP brain and ex vivo performance in pediatric biological fluids and brain tissue. FIG. 15A provides a scheme depicting ultrasound-guided ICV injection of LNPs in a 0.61G Macaca fascicularis. FIG. 15B provides ultrasound images (arrow points to needle) pre- and post-ICV injection of C3.GFP LNPs (dose=1 mg/kg total mRNA) in fetal macaque. FIG. 15C depicts GFP immunohistochemistry on brain sections from C3 LNP.GFP-treated and negative control animals. Scale bars: 100 μm (10×). FIG. 15D depicts size and PDI measurements of C3.MPS LNPs incubated in PBS, human CSF, or human serum. **** p<0.0001 by one-way ANOVA with Tukey's multiple comparisons test. FIG. 15E depicts size measurements of C3.MPS LNPs incubated in human CSF over a 7-day time course. FIG. 15F depicts GFP positivity in two patient-derived primary cell lines enriched for neurons after C3.GFP LNP treatment. FIG. 15G depicts viability after C3.GFP LNP treatment in both patient-derived cell lines. FIG. 15H depicts NGS results in patient-derived precision cut brain slices treated with C3.MPS LNPs normalized to control (PBS). Outliers were detected using Grubb's test and removed from analysis; minimum n=3 per treatment group; error bars represent SEM. FIG. 15I depicts the size and PDI measurements of C3.MPS LNPs incubated in human CSF at increasing percentages of CSF (v/v; volume CSF/volume LNP). FIG. 15J depicts the size and PDI measurements of C3.MPS LNPs incubated in human serum at increasing percentages of serum (v/v; volume human serum/volume LNP). FIG. 15K depicts zeta potential measurements of C3.MPS LNPs incubated in human CSF at increasing percentages of CSF (v/v). **** p<0.0001 by one way ANOVA with post hoc Dunnett's test compared to positive control (0%).

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

Delivery of mRNA-based therapeutics to the perinatal brain holds great potential in treating congenital brain diseases. However, non-viral delivery platforms that facilitate nucleic acid delivery in this environment have yet to be rigorously studied. In one aspect, the present disclosure describes a screen a diverse library of ionizable lipid nanoparticles (LNPs) via intracerebroventricular (ICV) injection in both fetal and neonatal mice and the resultant identification of an LNP formulation with greater functional mRNA delivery in the perinatal brain than an FDA-approved industry standard LNP. Following in vitro optimization of the top-performing LNP (i.e., C3 LNP) for co-delivery of an adenine base editing platform, the biochemical phenotype of a lysosomal storage disease in the neonatal mouse brain is improved, proof-of-principle mRNA brain transfection in vivo in a fetal NHP model is exhibited, and the translational potential of C3 LNPs ex vivo in human patient-derived brain tissues is demonstrated. These LNPs may provide a clinically translatable platform for in utero and postnatal gene editing in the brain.

A combined strength and limitation of LNPs is that each nucleic acid delivery application often requires careful engineering. Indeed, as described herein, the top-performing LNP following in utero ICV mRNA delivery was in the bottom quartile of LNPs screened via in utero intravascular mRNA delivery. LNPs containing the C3 ionizable lipid were small (<100 nm), monodisperse (<0.20 PDI) and well-encapsulated (>90%), which matches known trends for successful LNP-mediated nucleic acid delivery. Notably, LNPs with C3 ionizable lipid produced LNPs with the lowest pKₐ (5.05) among the LNP library. Previous reports have suggested that high pKₐ values (>5.5) confer efficacy, since LNPs protonate and escape the endosome prior to lysosomal acidification and endosomal internalization, transport, and escape are pH dependent processes. However, given that fetal serum pH is less than adult serum pH and CSF pH is less than serum pH, low pKₐ ionizable lipids with acceptable size and encapsulation properties may be most suitable for delivery to the perinatal CNS.

As described herein, the C3 LNP formulation was selected for in vitro optimization in neural-origin cells that mimic the intended in vivo application. Application of C3 LNPs to deliver gene editing platforms in vivo resulted in low levels of gene modulation (˜1-2%). Notably, LNP-mediated delivery of both Cre mRNA and base editing cargos resulted in similar levels of gene modulation, suggesting strong dependence on the delivery vector. Of note, histological studies in both small and large animal models revealed delivery of the mRNA cargo to brain ventricular tissues near the site of LNP ICV injection with low tissue perfusion to internal structures, limiting the application of LNP platforms in disseminated brain diseases. Without wishing to be bound by theory, strategies to improve tissue penetration of LNPs, such as modulation of LNP size or PEG density, may improve whole brain LNP-mediated gene editing.

Despite low LNP-mediated base editing in the exemplified disease model, a partial restoration of deficient IDUA enzyme activity (7-11% of normal) in the brain was observed, likely due to targeting of the high protein producing cerebral ventricular lining. Biochemical studies in patients with MPS-IH imply that a restoration of greater than 1-2% of IDUA activity may be sufficient to transition from a phenotype of the more severe Hurler's syndrome to the milder Scheie syndrome. IDUA enzyme activity in the brain was also greater following LNP-mediated local ICV injection in comparison to AAV-mediated systemic injection of the same base editing platform in prior work.

However, no mRNA delivery above baseline was observed in the liver or gonads following ICV delivery of the C3 LNP in the mouse studies, suggesting confinement of delivery to the brain. Thus, while the LNP platform developed in this study could be applied to mitigate the neurophenotype of MPS-IH and potentially other CNS genetic disorders, an additional systemic delivery approach would be required to facilitate correction of the multiorgan disease associated with MPS-IH.

For clinical application of a delivery technology targeting the perinatal brain, safety is paramount to prevent unintended clinical side-effects. From a genomic perspective, the LNP-mediated base editing strategy disclosed herein did not demonstrate significant editing in the gonads, implying limited risk to the germline. Although a limited number of cytokines were elevated in the serum following ICV delivery of C3 LNPs, cytokine levels were reduced compared to those generated following ICV injection of an FDA-approved LNP carrying the same base editing platform. Injection of C3 LNPs in the mouse neonate, which immunologically recapitulates a mid-gestation human fetus, did not elicit an anti-PEG IgM antibody response, in contrast to that seen following injection of the same LNPs in the adult. This highlights the opportunity for repeat LNP injections to boost longitudinal therapeutic efficacy. Although encouraging, additional safety analyses of the presently disclosed LNP base editing platform specific to both the therapeutic editing strategy and the LNP delivery platform are required prior to translation, including unbiased genomic off-target analyses, long-term anti-PEG IgG response, and evaluation of long-term systemic organ toxicity.

The eventual clinical application of in utero CNS directed gene editing via local LNP delivery to the brain would involve a minimally invasive ultrasound-guided injection into the ventricles or cisterna magna. To validate the feasibility of this approach, LNP-mediated mRNA delivery to the monkey brain ventricular lining was demonstrated and a similar delivery profile to that observed in the mouse brain was noted. Given that human fluids and tissues have distinct physiochemical properties, the performance of C3 LNPs in patient-derived samples was evaluated. Importantly, in contrast to that seen following incubation with human serum, C3 LNPs did not aggregate in human CSF and had a negative zeta potential, which minimizes potential adhesion to a brain extracellular matrix rich in negatively charged proteins. The stability of these LNPs within the medium in which they would ultimately be delivered clinically supports their translational potential.

Finally, using precision cut slices of human brain tissue, LNP-mediated base editing of the gene implicated in MPS-IH in primary neural-origin tissue was demonstrated. The present disclosure provides the first demonstration of base editing facilitated by a non-viral delivery carrier in primary human CNS tissue. As such, this experiment both validates the clinical feasibility of utilizing LNPs to base edit the human brain and serves as a foundation for future optimization in patient-derived tissues.

Delivery of mRNA-based therapeutics to the perinatal brain holds great potential in treating congenital brain disorders that currently lack sufficient therapeutic options. Although a number of studies have evaluated viral vector targeting of the CNS, non-viral mediated delivery of mRNA therapeutics to the CNS has been less robustly studied and offers potential safety advantages over viral vector delivery approaches. In one aspect, the present disclosure provides an LNP platform to meet this need, demonstrating the safety of this platform and therapeutic base editing in a small animal model of a human disease and support the translational potential of this approach via studies in the fetal nonhuman primate model and patient-derived brain tissues. These LNPs offer a translatable delivery platform for in utero CNS-directed gene therapy and gene editing.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C═C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein, refer to a divalent radical of the moiety corresponding to the base group (e.g., heteroalkyl, cycloalkyl, and/or heterocycloalkyl). A divalent radical possesses two open valencies at any position(s) of the group, wherein each radical may be on a carbon atom or heteroatom. Thus, the divalent radical may form a single bond to two distinct atoms or groups, or may form a double bond with one atom.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “amine” as used herein refers to primary, secondary, and tertiary amines having, e.g., the formula N (group) 3 wherein each group can independently be H or non-H, such as alkyl, aryl, and the like. Amines include but are not limited to R —NH₂, for example, alkylamines, arylamines, alkylarylamines; R²NH wherein each R is independently selected, such as dialkylamines, diarylamines, aralkylamines, heterocyclylamines and the like; and R³N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR², —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2- , 3- , 4-, 5-, or 6-positions of the phenyl ring, or a naphthyl group substituted at any one or more of 2- to 8-positions thereof.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, C₁₈ alkyl chains, ether linkages between the head group and alkyl chains, and 0 to 3 double bonds. Such lipids include, e.g., DSDMA, DLinDMA, DLenDMA, and DODMA.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichlorocthyl, 1,2-dichloroethyl, 1,3-dibromo-3,3-difluoropropyl, perfluorobutyl, and the like.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz [b,f]azepine (5H-dibenz [b,f]azepin-1-yl, 5H-dibenz [b,f]azepine-2-yl, 5H-dibenz [b,f]azepine-3-yl, 5H-dibenz [b,f]azepine-4-yl, 5H-dibenz [b,f]azepine-5-yl), 10,11-dihydro-5H-dibenz [b,f]azepine (10,11-dihydro-5H-dibenz [b,f]azepine-1-yl, 10,11-dihydro-5H-dibenz [b,f]azepine-2-yl, 10,11-dihydro-5H-dibenz [b,f]azepine-3-yl, 10,11-dihydro-5H-dibenz [b,f]azepine-4-yl, 10,11-dihydro-5H-dibenz [b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2- , 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b}) hydrocarbyl, wherein a and b are integers and mean having any of a to b number of carbon atoms. For example, (C₁-C₄) hydrocarbyl means the hydrocarbyl group can be methyl (C₁), ethyl (C₂), propyl (C3), or butyl (C₄), and (C₀-C_{b}) hydrocarbyl means in certain embodiments there is no hydrocarbyl group.

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, cosinophils, basophils, and the like.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “monovalent” as used herein refers to a substituent connecting via a single bond to a substituted molecule. When a substituent is monovalent, such as, for example, F or Cl, it is bonded to the atom it is substituting by a single bond.

The term “mRNA” or “messenger RNA” as used herein refers to a ribonucleic acid sequences which encodes a peptide or protein. In certain embodiments, the mRNA may comprise a “transcript” that is produced by using a DNA template and encodes a peptide or protein. Typically, mRNA comprises 5′-UTR, protein coding region and 3′-UTR. mRNA can be produced by in vitro transcription from a DNA template. Methods of in vitro transcription are known to those of skill in the art. For example, various in vitro transfer kits are commercially available. According to the present invention, mRNA can be modified by further stabilizing modifications and cap formation in addition to the modifications according to the invention.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (PI, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

The term “sgRNA” as used herein refers to a single-guide RNA (i.e., a single, contiguous polynucleotide sequence) that essentially comprises a crRNA connected at its 3′ end to the 5′ end of a tracrRNA through a “loop” sequence (see, e.g., U.S. Published patent application No. 20140068797, which is hereby incorporated herein by reference in its entirety). sgRNA interacts with a cognate Cas protein essentially as described for tracrRNA/crRNA polynucleotides, as described herein. Similar to crRNA, sgRNA has a spacer, a region of complementarity to a potential DNA target sequence, adjacent a second region that forms base-pair hydrogen bonds that form a secondary structure, typically a stem structure. The term includes truncated single-guide RNAs (tru-sgRNAs) of approximately 17-18 nt. The term also encompasses functional miniature sgRNAs with expendable features removed, but that retain an essential and conserved module termed the “nexus” located in the portion of sgRNA that corresponds to tracrRNA (not crRNA).

The terms “spacer” or “spacer element” as used herein with reference to a crRNA or sgRNA, refers to the polynucleotide sequence that can specifically hybridize to a target nucleic acid sequence. The spacer element interacts with the target nucleic acid sequence through hydrogen bonding between complementary base pairs (i.e., paired bases). A spacer element binds to a selected DNA target sequence. Accordingly, the spacer element is a DNA target-binding sequence. The spacer element determines the location of Cas protein's site-specific binding and endonucleolytic cleavage. Spacer elements range from 17-to-84 nucleotides in length, depending on the Cas protein with which they are associated, and have an average length of 36 nucleotides. For example, for SpyCas9, the functional length for a spacer to direct specific cleavage is typically about 12-25 nucleotides. Variability of the functional length for a spacer element is known in the art, as indicated in U.S. Published Patent Application No. 2014/0315985, which is incorporated herein by reference in its entirety.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O) C(O)R, C(O) CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH) N(R) 2, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In some embodiments, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

### Lipids

In one aspect, the present disclosure provides an ionizable lipid of Formula (I), or a salt, solvate, stereoisomer, or isotopologue thereof:

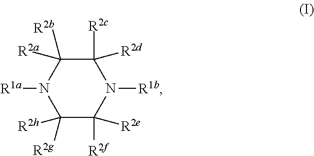

wherein:
R^{1a} and R^{1b} are each independently

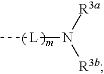

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴) (R⁵), -(optionally substituted C₁-C₆ alkylenyl)-C(═O)R⁴, -(optionally substituted C₁-C₆ alkylenyl)-(R⁴), —C(═O)OR⁴, —C(—O)N(R⁴) (R⁵), —C(═O)R⁴, and R⁴,
   wherein no more than one of each occurrence of R^{3a}, R^{3b}, and R^{3c} is H;

R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of (optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2e}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L is —CH₂—. In certain embodiments, L is —(CH₂)₂—. In certain embodiments, L is —(CH₂)₃—. In certain embodiments, L is —(CH₂)₁₀—. In certain embodiments, L is —(CH₂)₂O—. In certain embodiments, L is —(CH₂)₃O—. In certain embodiments, L is —CH₂CH(OR⁵) CH₂—. In certain embodiments, L is —(CH₂) 2NR^{3c}—. In certain embodiments, L is

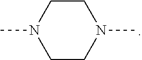

In certain embodiments, L is In certain embodiments, L is

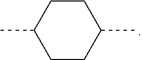

In certain embodiments, L is

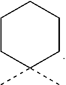

For instances of L which are asymmetric (e.g., —(CH₂)₃O—) it is understood that the present disclosure encompasses both possible orientations (e.g., —(CH₂)₃O— and —O(CH₂)₃—). In certain embodiments, the ionizable lipid of Formula (I) is:

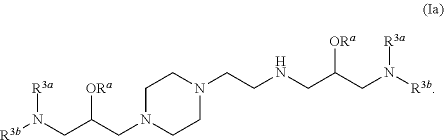

In certain embodiments, the ionizable lipid of

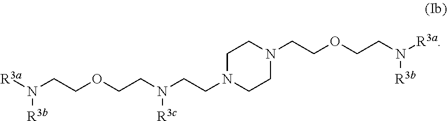

Formula (I) is:

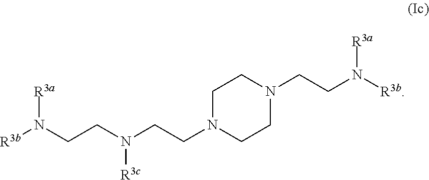

In certain embodiments, the ionizable lipid of Formula (I) is:

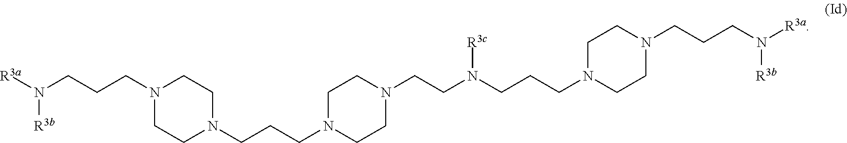

In certain embodiments, the ionizable lipid of Formula (I) is:

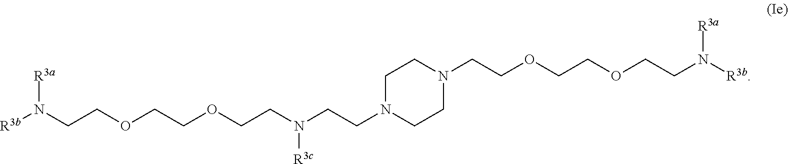

In certain embodiments, the ionizable lipid of Formula (I) is:

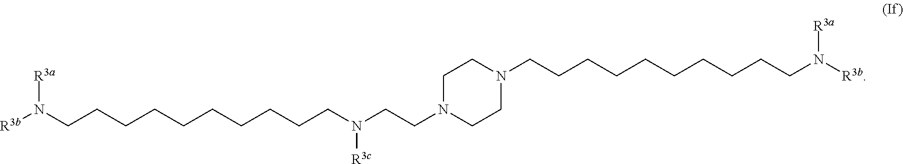

In certain embodiments, the ionizable lipid of Formula (I) is:

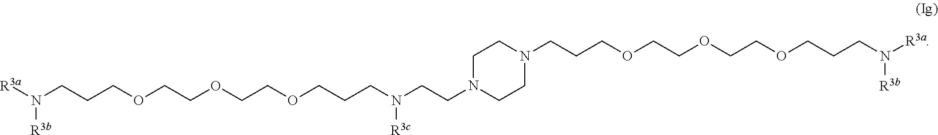

In certain embodiments, the ionizable lipid of Formula (I) is:

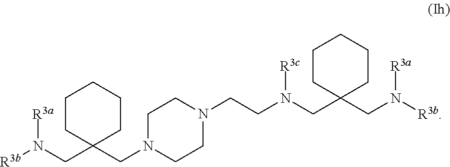

In certain embodiments, the ionizable lipid of Formula (I) is:

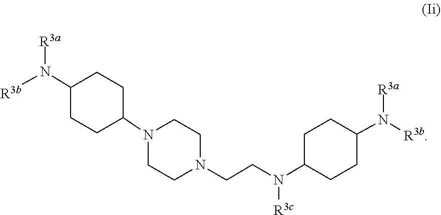

In certain embodiments, R^{3a} is H. In certain embodiments, R^{3a} is —CH₂CH(OH) (optionally substituted C₁-C₂₈ alkyl). In certain embodiments, R^{3a} is —CH₂CH(OH) (optionally substituted C₂-C₂₈ alkenyl). In certain embodiments, R^{3a} is —CH₂CH₂C(═O)O (optionally substituted C₁-C₂₈ alkyl). In certain embodiments, R^{3a} is —CH₂CH₂C(═O) NH (optionally substituted C₁-C₂₈ alkyl). In certain embodiments, R^{3b} is H. In certain embodiments, R^{3b} is —CH₂CH(OH) (optionally substituted C₁-C₂₈ alkyl). In certain embodiments, R^{3b} is —CH₂CH(OH) (optionally substituted C₂-C₂₈ alkenyl). In certain embodiments, R^{3b} is —CH₂CH₂C(═O)O (optionally substituted C₁-C₂₈ alkyl). In certain embodiments, R^{3b} is —CH₂CH₂C(═O) NH (optionally substituted C₁-C₂₈ alkyl). In certain embodiments, R^{3c} is H. In certain embodiments, R^{3c} is —CH₂CH(OH) (optionally substituted C₁-C₂₈ alkyl). In certain embodiments, R^{3c} is —CH₂CH(OH) (optionally substituted C₂-C₂₈ alkenyl). In certain embodiments, R^{3c} is —CH₂CH₂C(═O)O (optionally substituted C₁-C₂₈ alkyl). In certain embodiments, R^{3c} is —CH₂CH₂C(═O) NH (optionally substituted C₁-C₂₈ alkyl).

In certain embodiments, R^{3a} is —CH₂CH(OH) (CH₂) 9CH₃. In certain embodiments, R^{3a} is —CH₂CH(OH) (CH₂) 1CH₃. In certain embodiments, R^{3a} is —CH₂CH(OH) (CH₂) 13CH₃. In certain embodiments, R^{3b} is —CH₂CH(OH) (CH₂) 9CH₃. In certain embodiments, R^{3b} is —CH₂CH(OH) (CH₂) 1CH₃. In certain embodiments, R^{3b} is —CH₂CH(OH) (CH₂) 13CH₃. In certain embodiments, R^{3c} is —CH₂CH(OH) (CH₂) 9CH₃. In certain embodiments, R^{3c} is —CH₂CH(OH)(CH₂)₁₁CH₃. In certain embodiments, R^{3c} is —CH₂CH(OH) (CH₂)₁₃CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO2, OH, N(R′)(R″), C(═O)R′, C(═O)OR′, OC(═O)OR′, C(—O)N(R′)(R″), S(═O)₂N(R′)(R″), N(R′)C(—O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the ionizable lipid of Formula (I) is:

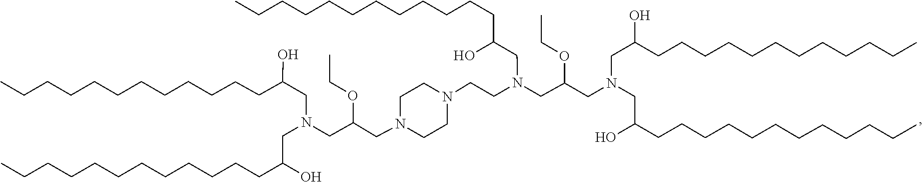 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]

### 1,1′-((3-(4-(2-((3-(bis(2-hydroxytetradecyl)amino)-2-ethoxypropyl) (2-hydroxytetradecyl)amino)ethyl) piperazin-1-yl)-2-ethoxypropyl) azanediyl)bis(tetradecan-2-ol) (C3)

Ionizable Lipids and/or Cationic Lipids

The scope of ionizable lipids contemplated for use in the present disclosure is not limited to ionizable lipids of Formula (I). In the lipid nanoparticles of the disclosure, the cationic lipid or ionizable lipid may comprise, e.g., one or more of the following: (6Z,9Z,28Z,31Z)-heptatriaconta-6,9,28,31-tetraen-19-yl 4-(dimethylamino) butanoate (DLinMC3DMA), [(4-hydroxybutyl) azanediyl|di (hexane-6,1-diyl)bis(2-hexyldecanoate) (ALC-0315), heptadecan-9-yl 8-{(2-hydroxyethyl) [6-oxo-6-(undecyloxy) hexyl]amino} octanoate (SM-102), 1,1′-[2-[4-[2-[[2-[bis(2-hydroxydodecyl)amino]ethyl](2-hydroxydodecyl)amino]ethyl]-1-piperazinyl]ethyl]imino]bis -2-dodecanol (C₁₂-200), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethylaminopropyl)-1,3]-dioxolane (D Lin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4- Nmethylpepiazino-[1,3]-dioxolane (DLin-K-MPZ), 2,2-dili-noleyl-4-dimethylaminomethyl-[1,3]-dioxolane (DLin-K-DMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino) propane (D Lin-MPZ), 3-(N,N-dilinoleylamino)-1,2-propanediol (D LinAP), 3-(N,N-dioleylamino)-1,2-propanedio (DOAP), 1,2-dilinoleyloxo-3-(2—N,N-dimethylamino) ethoxypropane (D Lin-EG-DMA), N,N-dioleyl-N,N-dimethylanrmonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy) propyl)-N,N,N-trimethylammonium chloride (DOTMA), N,N-distearyl-N,N-dimethylammonium bromide (DDAB), N-(1-(2,3-diolcoyloxy) propyl)-N,N, N-trimethylammonium chloride (DOTAP), 3-(N—(N′,N′dimethylaminocthane)-carbamoyl) cholesterol (DC-Chol), N-(1,2-dimyristyloxyprop-3-yl)-N,N-dimethyl-N-hydroxyethyl anrmonium bromide (DMRIE), 2,3-dioleyloxy-N-[2 (sperminc-carboxamidocthyl]-N,N-dimethy 1-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis -9,12-octadecadienoxy) propane (CLinDMA), 2-[5′-(cholest-5-en-3-beta-oxy)-3′-oxapentoxy)-3-dimethyl-1-(cis,cis -9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K—C₂-DMA (“XTC2”), or mixtures thereof. The ionizable lipids are not limited to those recited herein, and can further include ionizable lipids known to those skilled in the art, or described in PCT Application No. PCT/US2020/056255 and/or PCT Application No. PCT/US2020/056252, the disclosures of which are herein incorporated by reference in its entirety.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Application Publication No. US 2011/0256175, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.CI, DLin-TAP.CI, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. 20060240554, the disclosure of which is herein incorporated by reference in its entirety for all purposes.

### Non-Cationic Lipid

In the nucleic acid-lipid particles of the present disclosure, the non-cationic lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), diolcoylphosphatidylglycerol (DOPG), dipalmitoylphosphatidylglycerol (DPPG), iolcoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S—NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (C₁₈), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly (ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly (ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Lipid Nanoparticle (LNP) Compositions

In one aspect, the present disclosure provides a lipid nanoparticle (LNP) composition.

In certain embodiments, the LNP comprises at least one ionizable lipid.

In certain embodiments, the LNP comprises at least one helper lipid.

In certain embodiments, the LNP comprises cholesterol.

In certain embodiments, the LNP comprises at least one conjugated lipid.

In certain embodiments, the LNP comprises at least one nucleic acid cargo comprising at least one mRNA and at least one sgRNA.

In certain embodiments, the nucleic acid cargo are at least partially encapsulated in the LNP.

In certain embodiments, the at least one ionizable lipid comprises an ionizable lipid of Formula (I), or a salt, solvate, stereoisomer, or isotopologue thereof:

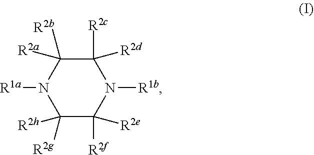

wherein:
R^{1a} and R^{1b} are each independently

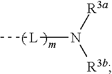

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴) (R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴) (R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of (optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L is —CH₂—. In certain embodiments, L is —(CH₂)₂—. In certain embodiments, L is —(CH₂)₃—. In certain embodiments, L is —(CH₂)₁₀—. In certain embodiments, L is —(CH₂)₂O—. In certain embodiments, L is —(CH₂)₃O—. In certain embodiments, L is —CH₂CH(OR⁵)CH₂—. In certain embodiments, L is —(CH₂)₂NR^{3c}—. In certain embodiments, L is

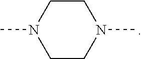

In certain embodiments, L is

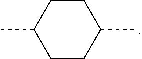

In certain embodiments, L is

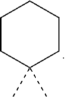

For instances of L which are asymmetric (e.g., —(CH₂)₃O—) it is understood that the present disclosure encompasses both possible orientations (e.g., —(CH₂)₃O— and —O(CH₂)₃—). In certain embodiments, the ionizable lipid of Formula (I) is:

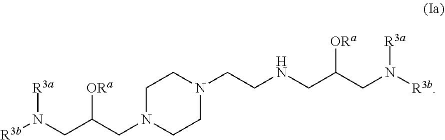

In certain embodiments, the ionizable lipid of Formula (I) is:

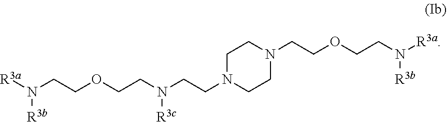

In certain embodiments, the ionizable lipid of Formula (I) is:

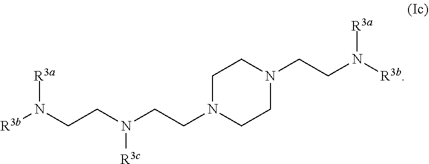

In certain embodiments, the ionizable lipid of Formula (I) is:

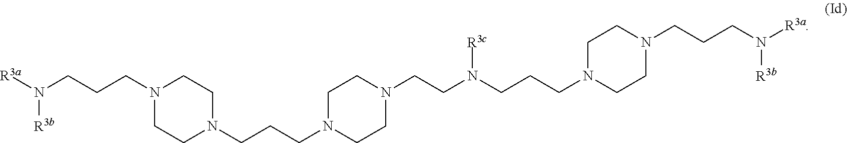

In certain embodiments, the ionizable lipid of Formula (I) is:

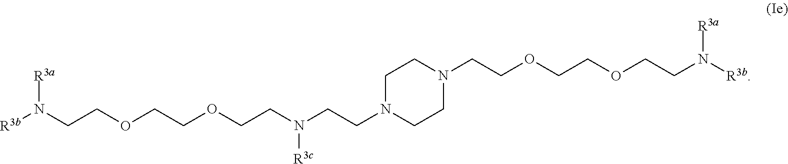

In certain embodiments, the ionizable lipid of Formula (I) is:

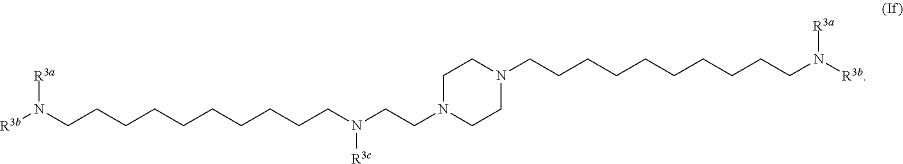

In certain embodiments, the ionizable lipid of Formula (I) is:

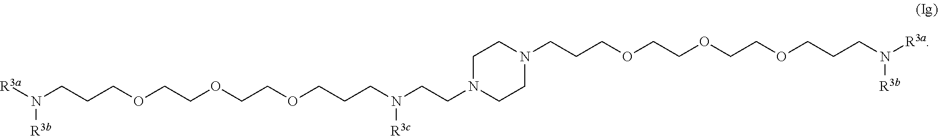

In certain embodiments, the ionizable lipid of Formula (I) is:

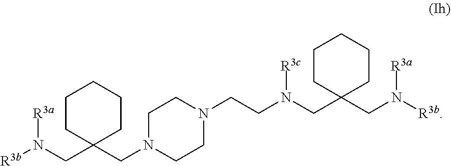

In certain embodiments, the ionizable lipid of Formula (I) is:

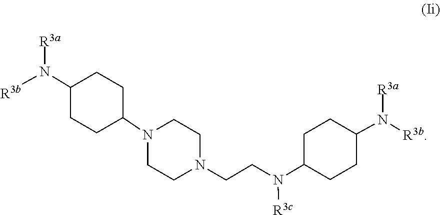

In certain embodiments, R^{3a} is H. In certain embodiments, R^{3a} is —CH₂CH(OH) (optionally substituted C₁-C₂₈ alkyl). In certain embodiments, R^{3a} is —CH₂CH(OH) (optionally substituted C₂-C₂₈ alkenyl). In certain embodiments, R^{3a} is —CH₂CH₂C(═O)O (optionally substituted C₁-C₂₈ alkyl). In certain embodiments, R^{3a} is —CH₂CH₂C(═O) NH (optionally substituted C₁-C₂₈ alkyl). In certain embodiments, R^{3b} is H. In certain embodiments, R^{3b} is —CH₂CH(OH) (optionally substituted C₁-C₂₈ alkyl). In certain embodiments, R^{3b} is —CH₂CH(OH) (optionally substituted C₂-C₂₈ alkenyl). In certain embodiments, R^{3b} is —CH₂CH₂C(═O)O (optionally substituted C₁-C₂₈ alkyl). In certain embodiments, R^{3b} is —CH₂CH₂C(═O) NH (optionally substituted C₁-C₂₈ alkyl). In certain embodiments, R^{3c} is H. In certain embodiments, R^{3c} is —CH₂CH(OH) (optionally substituted C₁-C₂₈ alkyl). In certain embodiments, R^{3c} is —CH₂CH(OH) (optionally substituted C₂-C₂₈ alkenyl). In certain embodiments, R^{3c} is —CH₂CH₂C(═O)O (optionally substituted C₁-C₂₈ alkyl). In certain embodiments, R^{3c} is —CH₂CH₂C(═O) NH (optionally substituted C₁-C₂₈ alkyl).

In certain embodiments, R^{3a} is —CH₂CH(OH) (CH₂) 9CH₃. In certain embodiments, R^{3a} is —CH₂CH(OH) (CH₂) 11CH₃. In certain embodiments, R^{3a} is —CH₂CH(OH) (CH₂) 13CH₃. In certain embodiments, R^{3b} is —CH₂CH(OH) (CH₂) 9CH₃. In certain embodiments, R^{3b} is —CH₂CH(OH) (CH₂) 1CH₃. In certain embodiments, R^{3b} is —CH₂CH(OH) (CH₂) 13CH₃. In certain embodiments, R^{3c} is —CH₂CH(OH) (CH₂) 9CH₃. In certain embodiments, R³° is —CH₂CH(OH) (CH₂) 11CH₃. In certain embodiments, R^{3c} is —CH₂CH(OH) (CH₂) 13CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the ionizable lipid of Formula (I) is:

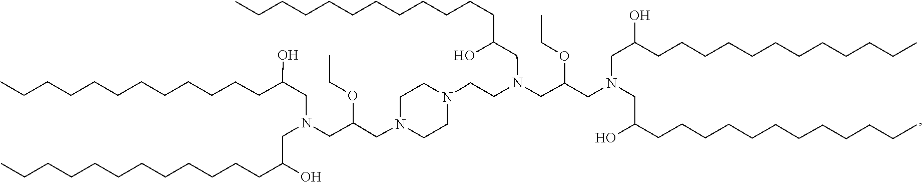 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]

1,1′-((3-(4-(2-((3-(bis(2-hydroxytetradecyl)amino)-2-ethoxypropyl) (2-hydroxytetradecyl)amino)ethyl) piperazin-1-yl)-2-ethoxypropyl) azanediyl)bis(tetradecan-2-ol) (C3).

In certain embodiments, the at least one ionizable lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises less than about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises more than about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises about 35 mol % of the LNP.

In certain embodiments, the helper lipid comprises dioleoylphosphatidylethanolamine (DOPE) and distearoylphosphatidylcholine (DSPC). In certain embodiments, the helper lipid is dioleoylphosphatidylethanolamine (DOPE). In certain embodiments, the helper lipid is distearoylphosphatidylcholine (DSPC).

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, or about 25 mol % of the LNP. In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, or about 25 mol % of the LNP. In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, or about 25 mol % of the LNP.

In certain embodiments, the at least one helper lipid comprises about 16 mol % of the LNP. In certain embodiments, the at least one helper lipid comprises about 14.5 mol % of the LNP. In certain embodiments, the at least one helper lipid comprises about 13 mol % of the LNP. In certain embodiments, the at least one helper lipid comprises about 11.5 mol % of the LNP. In certain embodiments, the at least one helper lipid comprises about 10 mol % of the LNP.

In certain embodiments, the LNP comprises about 16 mol % DOPE.

In certain embodiments, cholesterol comprises about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol comprises less than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol comprises more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol comprises about 46.5 mol % of the LNP.

In certain embodiments, the at least one conjugated lipid comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises about 2.5 mol % of the LNP.

In certain embodiments, the at least one conjugated lipid comprises a polyethylene glycol (PEG)-conjugated lipid. In certain embodiments, the at least one conjugated lipid comprises C14-PEG. In certain embodiments, C14-PEG comprises:

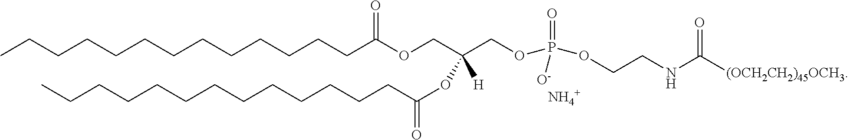

In certain embodiments, the LNP has a ratio of mRNA to sgRNA of about 0.1:1, 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, or about 1:0.1 (mRNA:sgRNA). In certain embodiments, the LNP has a ratio of mRNA to sgRNA of less than about 0.1:1, 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, or about 1:0.1 (mRNA:sgRNA). In certain embodiments, the LNP has a ratio of mRNA to sgRNA of more than about 0.1:1, 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, or about 1:0.1 (mRNA:sgRNA). In certain embodiments, the LNP has a ratio of mRNA to sgRNA of about 1:10, 2:10, 3:10, 4:10, 5:10, 6:10, 7:10, 8:10, 9:10, 10:10, 10:9, 10:8, 10:7, 10:6, 10:5, 10:4, 10:3, 10:2, or about 10:1 (mRNA:sgRNA). In certain embodiments, the LNP has a ratio of mRNA to sgRNA of less than about 1:10, 2:10, 3:10, 4:10, 5:10, 6:10, 7:10, 8:10, 9:10, 10:10, 10:9, 10:8, 10:7, 10:6, 10:5, 10:4, 10:3, 10:2, or about 10:1 (mRNA:sgRNA). In certain embodiments, the LNP has a ratio of mRNA to sgRNA of more than about 1:10, 2:10, 3:10, 4:10, 5:10, 6:10, 7:10, 8:10, 9:10, 10:10, 10:9, 10:8, 10:7, 10:6, 10:5, 10:4, 10:3, 10:2, or about 10:1 (mRNA:sgRNA). In certain embodiments, the LNP has a ratio of mRNA to sgRNA of about 3:1. In certain embodiments, the ratio is a molar ratio. In certain embodiments, the ratio is by weight.

In certain embodiments, the LNP has a molar ratio of (a):(b):(c):(d) of about 35:16:46.5:2.5. In certain embodiments, the LNP has a molar ratio of (a):(b):(c):(d) of less than about 35:16:46.5:2.5. In certain embodiments, the LNP has a molar ratio of (a):(b):(c):(d) of more than about 35:16:46.5:2.5.

In certain embodiments, the LNP has a ratio of ionizable lipid to total nucleic acid (ionizable lipid:mRNA+sgRNA) ranging of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or about 30:1. In certain embodiments, the LNP has a ratio of ionizable lipid to total nucleic acid (ionizable lipid:mRNA+sgRNA) ranging of less than about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or about 30:1. In certain embodiments, the LNP has a ratio of ionizable lipid to total nucleic acid (ionizable lipid:mRNA+sgRNA) ranging of more than about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or about 30:1. In certain embodiments, the LNP has a ratio of ionizable lipid to total nucleic acid (ionizable lipid:mRNA+sgRNA) of about 10:1. In certain embodiments, the ratio is a molar ratio. In certain embodiments, the ratio is by weight.

In certain embodiments, the mRNA encodes a base editor. In certain embodiments, the mRNA encodes each component of a base-editing complex. In certain embodiments, the base editor comprises an adenine base editor and/or adenine base editing complex. In certain embodiments, the base editor comprises a cytosine editor and/or cytosine editing complex.

In certain embodiments, the sgRNA is specific to a mutated site in the genome of a subject which is correlated with a neurological disease and/or disorder. In certain embodiments, the nucleic acid cargo is selectively delivered to the brain of a subject.

### Cargo

In one aspect, the present disclosure relates to LNPs comprising at least one cargo molecule at least partially encapsulated therein. In certain embodiments, the at least one cargo is fully encapsulated therein. In certain embodiments, the cargo comprises at least one mRNA molecule and at least one sgRNA molecule. In certain embodiments, the at least one mRNA molecule and at least one sgRNA molecule are suitable for base-editing (i.e., mRNA encoding a base editor and/or polypeptides encoding each component of a base editor complex; and a sgRNA suitable to guide the base editor and/or base editing complex to an appropriate site for base editing).

### Base-Editing

In certain embodiments of the present disclosure, a subject is administered a base-editor and/or base-editing complex for in utero gene editing. In certain embodiments, the base-editor and/or base-editing complex comprises an adenine base-editor (ABE). In certain embodiments, the base-editor and/or base-editing complex comprises a cytosine base-editor (CBE).

In certain embodiments, the ABE or CBE complex comprises a catalytically impaired Streptococcus pyogenes Cas9 (SpCas9) protein, unable to make DSBs, fused to either a cytosine deaminase domain from a nucleic acid-editing protein or a modified tRNA adenosine deaminase. The SpCas9 and sgRNA tether the base editor at the genome target site, and the cytosine deaminase converts a nearby cytosine into uracil and, ultimately, thymine (resulting in either C T or G A changes in the coding sequence of a gene, depending on which strand is targeted). The cytosine deaminase can introduce nonsense mutations in a site-specific fashion. Alternatively, the adenine deaminase converts a nearby adenine into inosine and, ultimately, guanine and can correct a disease-causing G A mutation. Unlike HDR, base editing does not require proliferating cells to efficiently introduce mutations. Non-limiting examples of ABE and/or CBE complexes are described in International Application No. PCT/US2021/071011, which is incorporated herein by reference in its entirety.

Methods for selecting and preparing sgRNA molecules suitable for binding to an appropriate mutated site in a genome are known to those of ordinary skill in the art, and specific mutations underlying a number of genetic diseases, including neurological diseases, characterized by a single nucleotide mutation are known to those of ordinary skill in the art. Non-limiting examples single nucleotide mutations underlying genetic diseases and/or sgRNA sequences suitable for binding such sequences in the genome of a subject are described in International Application No. PCT/US2021/071011, which is incorporated herein by reference in its entirety.

In certain embodiments, the cargo comprises at least one selected from the group consisting of a nucleic acid molecule, small molecule, protein, therapeutic agent, antibody, and any combinations thereof.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In certain embodiments, the composition of the invention comprises an in vitro transcribed (IVT) RNA molecule. For example, in certain embodiments, the composition of the invention comprises an IVT RNA molecule which encodes an agent. In certain embodiments, the IVT RNA molecule of the present composition is a nucleoside-modified mRNA molecule. In certain embodiments, the agent is for targeting an immune cell to a pathogen or a tumor cell of interest. In certain embodiments, the IVT RNA molecule encodes a chimeric antigen receptor (CAR).

In some embodiments, the CAR is specific for binding to one or more antigens. In some embodiments, the antigen comprises at least one viral antigen, a bacterial antigen, a fungal antigen, a parasitic antigen, an influenza antigen, a tumor-associated antigen, a tumor-specific antigen, or any combination thereof.

However, the present invention is not limited to any particular agent or combination of agents. In certain embodiments, the composition comprises an adjuvant. In certain embodiments, the composition comprises a nucleic acid molecule encoding an adjuvant. In certain embodiments, the composition comprises a nucleoside-modified RNA encoding an adjuvant.

In certain embodiments, the composition comprises at least one RNA molecule encoding a combination of at least two agents. In certain embodiments, the composition comprises a combination of two or more RNA molecules encoding a combination of two or more agents.

In certain embodiments, the present invention provides a method for inducing an immune response in a subject. For example, the method can be used to provide immunity in the subject against a virus, bacteria, fungus, parasite, cancer, or the like. In some embodiments, the method comprises administering to the subject a composition comprising one or more LNP molecule formulated for in vivo targeting of an immune cell comprising one or more RNA encoding at least one antigen, an adjuvant, or a combination thereof.

In certain embodiments, the present invention provides a method for gene editing of an immune cell of a subject. For example, the method can be used to provide one or more component of a gene editing system (e.g., a component of a CRISPR system) to an immune cell of a subject. In some embodiments, the method comprises administering to the subject a composition comprising one or more ionizable LNP molecule formulated for targeted T cell delivery comprising one or more nucleoside-modified RNA molecule for gene editing.

In certain embodiments, the method comprises administration of the composition to a subject. In certain embodiments, the method comprises administering a plurality of doses to the subject. In some embodiments, the method comprises administering a single dose of the composition, where the single dose is effective in delivery of the target therapeutic agent.

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PTPN22 using RNAi technology.

In one aspect, the invention includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl-methyl-, thio- and other modified forms of adenine, cytidine, guanine, thymine, and uridinc.

In some embodiments, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In some embodiments, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis -dependent or independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3 ‘-terminus can be blocked with an aminoalkyl group. Other 3’ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the ICsQ. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,61 1, 5,753,613, 5,785,992, 6, 120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide Therapeutic Agents

In other related aspects, the therapeutic agent includes an isolated peptide that modulates a target. For example, In certain embodiments, the peptide of the invention inhibits or activates a target directly by binding to the target thereby modulating the normal functional activity of the target. In certain embodiments, the peptide of the invention modulates the target by competing with endogenous proteins. In certain embodiments, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His -tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

CAR agents

In certain embodiments, the mRNA molecule of the invention encodes a chimeric antigen receptor (CAR). In certain embodiments, the CAR comprises an antigen binding domain. In certain embodiments, the antigen binding domain is a targeting domain, wherein the targeting domain directs the T cell expressing the CAR to a specific cell or tissue of interest. For example, In certain embodiments, the targeting domain comprises an antibody, antibody fragment, or peptide that specifically binds to an expressed on a pathogenic organism or a tumor cell thereby directing the T cell expressing the CAR to a cell or tissue expressing the antigen.

In certain embodiments, the invention relates to an immune cell targeted LNP comprising an agent, wherein the agent comprises a nucleic acid sequence encoding a chimeric antigen receptor (CAR). In certain embodiments, agent comprises an mRNA molecule encoding a CAR. In certain embodiments, the agent comprises a modified nucleoside mRNA molecule encoding a CAR.

In various embodiments, the CAR can be a “first generation,” “second generation,” “third generation,” “fourth generation” or “fifth generation” CAR (see, for example, Sadelain et al., Cancer Discov. 3 (4): 388-398 (2013); Jensen et al., Immunol. Rev. 257:127-133 (2014); Sharpe et al., Dis. Model Mech. 8 (4): 337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3-chain, which is the primary transmitter of signals from endogenous T cell receptors (TCRs). “First generation” CARs can provide de novo antigen recognition and cause activation of both CD4+ and CD8+ T cells through their CD35 chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD32 signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1 (9): 1577-1583 (2012)).

“Third generation” CARs provide multiple co-stimulation, for example, by comprising both CD28 and 4-1BB domains, and activation, for example, by comprising a CD32 activation domain.

“Fourth generation” CARs provide co-stimulation, for example, by CD28 or 4-1BB domains, and activation, for example, by a CD3 signaling domain in addition to a constitutive or inducible chemokine component.

“Fifth generation” CARs provide co-stimulation, for example, by CD28 or 4-1BB domains, and activation, for example, by a CD32 signaling domain, a constitutive or inducible chemokine component, and an intracellular domain of a cytokine receptor, for example, IL-2RB.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is an scFv specific for binding to a surface antigen of a target cell of interest (e.g., a pathogen or tumor cell.)

### Combinations

In certain embodiments, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Cell Targeting Domain

In various embodiments of the invention, the LNP of the invention is conjugated to a targeting domain specific for binding to a receptor of a target cell.

In certain embodiments, the target cell is a stem cell. Exemplary stem cells that can be targeted by the compositions of the invention include, but are not limited to, hematopoietic stem cells and stem cells related to hematopoietic stem cells (e.g., myeloid stem cells and lymphoid stem cells.)

In certain embodiments, the target cell is a peripheral blood mononuclear cell (PBMC).

In one cell the target cell is an immune cell. Exemplary immune cells that can be targeted according by the compositions of the invention include, but are not limited to, T cells, B cells, NK cells, antigen-presenting cells, dendritic cells, macrophages, monocytes, neutrophils, cosinophils, and basophils. In certain embodiments, the immune cell is a T cell. In some embodiments, T cells that can be targeted using the compositions of the invention can be CD4+ or CD8+ and can include, but are not limited to, T helper cells (CD4+), cytotoxic T cells (also referred to as cytotoxic T lymphocytes, CTL; CD8− T cells), and memory T cells, including central memory T cells (TCM), stem memory T cells (TSCM), stem-cell-like memory T cells (or stem-like memory T cells), and effector memory T cells, for example, T_{EM} cells and T_{EMRA} (CD45RA+) cells, effector T cells, Th1 cells, Th2 cells, Th9 cells, Th17 cells, Th22 cells, Tfh (follicular helper) cells, T regulatory cells, natural killer T cells, mucosal associated invariant T cells (MAIT), and γδ T cells. Major T cell subtypes include TN (naive), T_{SCM} (stem cell memory), T_{CM} (central memory), T_{TM} (Transitional Memory), T_{EM} (Effector memory), and TTE (Terminal Effector), TCR-transgenic T cells, T-cells redirected for universal cytokine-mediated killing (TRUCK), Tumor infiltrating T cells (TIL), CAR-T cells or any T cell that can be used for treating, ameliorating, and/or preventing a disease or disorder.

In certain embodiments, the T cells of the invention are immunostimulatory cells, i.e., cells that mediate an immune response. Exemplary T cells that are immunostimulatory include, but are not limited to, T helper cells (CD4+), cytotoxic T cells (also referred to as cytotoxic T lymphocytes, CTL; CD8+ T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th 17 cells, Th22 cells, Tfh (follicular helper) cells, natural killer T cells, mucosal associated invariant T cells (MAIT), and γδ T cells.

In certain embodiments, the T cell targeting domain binds to CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R¹, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, or CCR7.

In certain embodiments, present invention relates to compositions comprising a combination of delivery vehicles conjugated to immune cell targeting domains for targeting multiple immune cells. In certain embodiments, the combination comprises two or more immune cell targeted delivery vehicles, targeting two or more immune cell antigens. In certain embodiments, the two or more immune cell antigens are selected from CDI, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R¹, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7. In certain embodiments, the combination comprises two or more T cell targeted delivery vehicles, targeting a surface antigen of a CD4+ T cell and a surface antigen of a CD8+ T cell. In certain embodiments, the combination comprises two or more T cell targeted delivery vehicles, targeting CD4 and CD8.

In certain embodiments, the targeting domain is conjugated to the LNP of the invention. Exemplary methods of conjugation can include, but are not limited to, covalent bonds, electrostatic interactions, and hydrophobic (“van der Waals”) interactions. In certain embodiments, the conjugation is a reversible conjugation, such that the delivery vehicle can be disassociated from the targeting domain upon exposure to certain conditions or chemical agents. In some embodiments, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In certain embodiments, the activated polymer conjugated lipid comprises a first coupling group capable of reacting with a second coupling group. In certain embodiments, the activated polymer conjugated lipid is an activated pegylated lipid. In certain embodiments, the first coupling group is bound to the lipid portion of the pegylated lipid. In some embodiments, the first coupling group is bound to the polyethylene glycol portion of the pegylated lipid. In certain embodiments, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, cpoxides, carbonates, anhydrides, sulfonyl chlorides, cyclooctyne, aldehydes, and sulfhydryl groups. In some embodiments, the first coupling group or second coupling group is selected from the group consisiting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as malcimide, pyridyl disulfide, or a haloacetyl. In certain embodiments, the first coupling group is a maleimide.

In certain embodiments, the second coupling group is a sulfhydryl group. The sulfhydryl group can be installed on the targeting domain using any method known to those of skill in the art. In certain embodiments, the sulfhydryl group is present on a free cysteine residue. In certain embodiments, the sulfhydryl group is revealed via reduction of a disulfide on the targeting domain, such as through reaction with 2-mercaptoethylamine. In certain embodiments, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone.

In certain embodiments, the targeting domain is conjugated to the LNP using maleimide conjugation.

### Targeting Domain

In certain embodiments, the composition comprises a targeting domain that directs the delivery vehicle to a target immune cell. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In various embodiments, the targeting domain binds to a cell surface molecule of a cell of interest. For example, in various embodiments, the targeting domain binds to a cell surface molecule of an endothelial cell, a stem cell, or an immune cell.

### Peptides

In certain embodiments, the targeting domain of the invention comprises a peptide. In certain embodiments, the peptide targeting domain specifically binds to a target of interest.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His -tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Antibodies

In certain embodiments, the targeting domain of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab) 2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Methods

In one aspect, the present disclosure provides a method of delivering a cargo to the brain of a subject in need thereof. In certain embodiments, the method comprises administering to the subject a therapeutically effective amount of at least one lipid nanoparticle (LNP).

In certain embodiments, the LNP comprises at least one ionizable lipid.

In certain embodiments, the LNP comprises at least one helper lipid.

In certain embodiments, the LNP comprises cholesterol.

In certain embodiments, the LNP comprises at least one conjugated lipid.

In certain embodiments, the LNP comprises at least one cargo molecule.

In certain embodiments, the at least one cargo molecule is at least partially encapsulated in the LNP.

In certain embodiments, the at least one ionizable lipid is the compound of Formula (I).

In certain embodiments, the cargo is at least one selected from the group consisting of a nucleic acid molecule, small molecule, protein, therapeutic agent, antibody, and any combinations thereof.

In certain embodiments, the cargo is a nucleic acid molecule. In certain embodiments, the nucleic acid molecule is a DNA molecule or a RNA molecule. In certain embodiments, the nucleic acid molecule is selected from the group consisting of mRNA, cDNA, pDNA, microRNA, siRNA, modified RNA, antagomir, antisense molecule, targeted nucleic acid, and any combination thereof.

In certain embodiments, the administration is intracerebrovenctricular (ICV). In certain embodiments, the administration is in utero. In certain embodiments, the subject is a mammal. In certain embodiments, the mammal is in an embryonic, fetal, perinatal, or neonatal stage of development. In certain embodiments, the mammal is a human.

In another aspect, the present disclosure provides a method of genome editing a mutated gene sequence associated with a disease or disorder in a subject in need thereof, the method comprising administering to the subject a therapeutically effective amount of at least one LNP of the present disclosure and/or a pharmaceutical composition thereof.

In certain embodiments, the disease or disorder is a neurological disease or disorder.

In certain embodiments, the neurological disease or disorder is at least one selected from the group consisting of a lysosomal storage disease, mitochondrial disease, Friedrich's ataxia, Huntington's disease.

In certain embodiments, the lysosomal storage disease is at least one selected from the group consisting of Farber disease, Krabbe disease, Fabry disease, Schindler disease, Sandhoff disease, Tay-Sachs disease, Gaucher disease, Niemann-Pick disease, Hurler syndrome, Scheie syndrome, Hurler-Scheie syndrome, Sanfilippo syndrome, Maroteaux-Lamy syndrome, Sly syndrome, hyaluronidase deficiency, sialidosis, I-cell disease, Santavuori-Haltia disease, Jansky-Bielschowsky disease, Batten-Spielmeyer-Vogt disease, Kufs disease, and Wolman disease.

In certain embodiments, the lysosomal storage disease is Hurler-Scheie syndrome.

In certain embodiments, the mutated gene comprises a G→A mutation in the Idua gene.

In certain embodiments, the administration is intracerebrovenctricular (ICV). In certain embodiments, the administration is in utero. In certain embodiments, the subject is a mammal. In certain embodiments, the mammal is in an embryonic, fetal, perinatal, or neonatal stage of development. In certain embodiments, the mammal is a human.

In another aspect, the present disclosure provides a method of treating, ameliorating, and/or preventing a lysosomal storage disease in a subject in need thereof, the method comprising administering to the subject a therapeutically effective amount of at least one LNP of the present disclosure and/or a pharmaceutical composition thereof.

In certain embodiments, the lysosomal storage disease is at least one selected from the group consisting of Farber disease, Krabbe disease, Fabry disease, Schindler disease, Sandhoff disease, Tay-Sachs disease, Gaucher disease, Niemann-Pick disease, Hurler syndrome, Scheic syndrome, Hurler-Scheie syndrome, Sanfilippo syndrome, Maroteaux-Lamy syndrome, Sly syndrome, hyaluronidase deficiency, sialidosis, I-cell disease, Santavuori-Haltia disease, Jansky-Bielschowsky disease, Batten-Spielmeyer-Vogt disease, Kufs disease, and Wolman disease.

In certain embodiments, the lysosomal storage disease is Hurler-Scheie syndrome.

In certain embodiments, the mutated gene comprises a G→A mutation in the Idua gene.

In certain embodiments, the administration is intracerebrovenctricular (ICV). In certain embodiments, the administration is in utero. In certain embodiments, the subject is a mammal. In certain embodiments, the mammal is in an embryonic, fetal, perinatal, or neonatal stage of development. In certain embodiments, the mammal is a human.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, rescaled erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for case of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μ g to about 6,000 mg, about 100 μg to about 5,500 mg, about 200 μ g to about 5,000 mg, about 400 μ g to about 4,000 mg, about 800 μ g to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intracerebroventricular, subcutaneous, intravenous, intraperitoneal, intramuscular, intrasternal injection, and kidney dialytic infusion techniques. In certain embodiments, the composition of the present disclosure is administered intracerebroventricularly.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXAMPLES

Various embodiments of the present application can be better understood by reference to the following Examples which are offered by way of illustration. The scope of the present application is not limited to the Examples given herein.

### Materials and Methods

Ionizable lipid synthesis

Ionizable lipids were prepared via nucleophilic addition as previously described, according to methods known to those of ordinary skill in the art. Briefly, epoxide-terminated alkyl chains (Avanti Polar Lipids) were reacted with polyamine cores (Enamine Inc., Monmouth Junction, NJ). The components were combined in a 4 mL amber vial equipped with a magnetic stir bar in the presence of an excess of epoxide precursors to saturate the amines. The crude product was transferred to a Rotavapor R-300 (BUCHI, New Castle, DE) for solvent evaporation, and the lipids were suspended in ethanol for use in the screening library.

mRNA synthesis

mRNA was produced using standard in vitro transcription methods. Briefly, luciferase, GFP, and ABE7.10 gene sequences were codon optimized, synthesized, and cloned into proprietary mRNA production plasmids. The m1Y′ UTP nucleoside modified mRNA was co-transcriptionally capped with a trinucleotide capl analogue (TriLink, San Diego, CA) and engineered to contain a 101 nucleotide-long poly (A) tail. Transcription was performed using MegaScript T7 RNA polymerase (Invitrogen, Waltham, MA), and mRNA was precipitated using lithium chloride prior to purification via cellulose chromatography. mRNAs were analyzed by agarose gel electrophoresis, sequenced, subjected to a standard J2 dot blot, assayed for INF induction in human monocyte derived dendritic cells, and stored at −80° C. for future use.

Cre recombinase mRNA fully substituted with 5-methoxyuridine was sourced from TriLink Biotechnologies (San Diego, CA) using their CleanCap® platform. sgRNAs were sourced from Integrated DNA Technologies (Coralville, IA) using their Alt-RTM platform. The mouse Idua gene targeting protospacer and PAM was 5′-ACTCTAGGCAGAGGTCTCAA|AGG-3′ (SEQ ID NO:1) as previously described in the literature. The protospacer and PAM to introduce the most common mutation in the W402X human Idua gene was 5′-CCAGAGCTGCTCCTCATCTG|CGG-3′ (SEQ ID NO:2).

### LNP Formulation

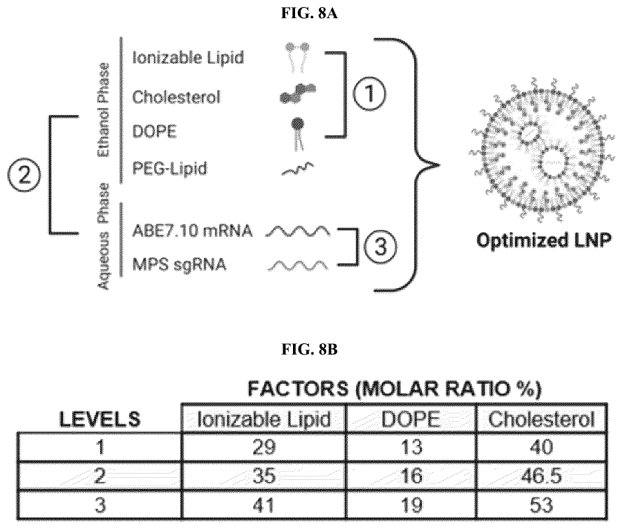

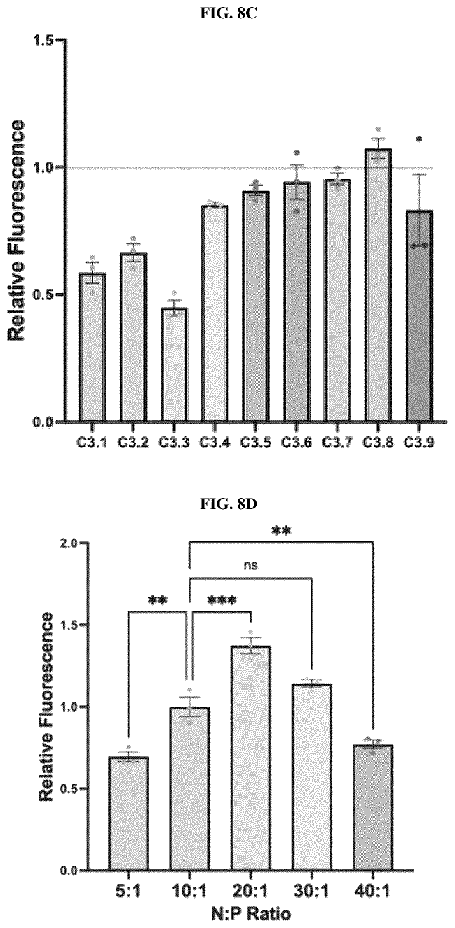

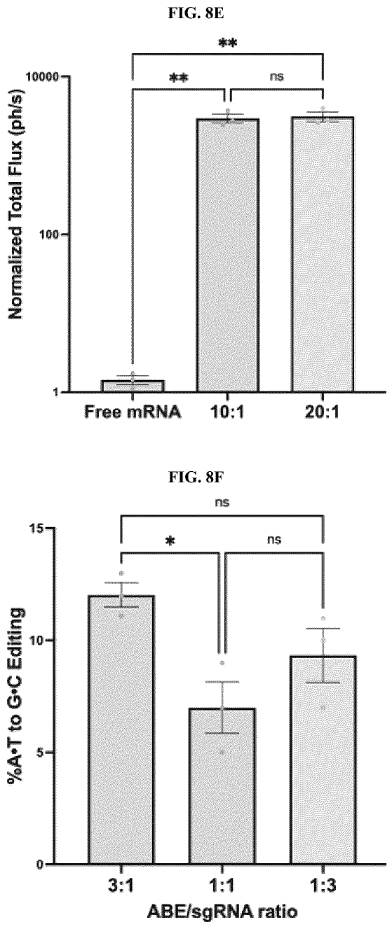

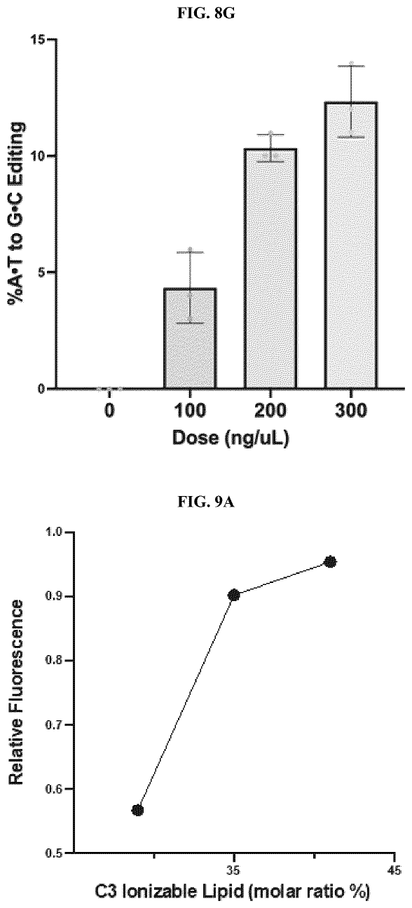

The ionizable lipids, prepared as described above, or DLin-MC3-DMA (MedChem Express, Monmouth Junction, NJ) were combined in an ethanol phase with cholesterol (Sigma-Aldrich, St. Louis, MO), DOPE (Avanti, Alabaster, AL), and 1,2-dimyristoyl-sn-glycero-3-phosphoethanolamine-N-[methoxy (polyethylene glycol)-2000](C14-PEG2000, Avanti) at a molar ratio of 35:46.5:16:2.5, respectively, or at molar ratios specified in FIG. 8B. For screening LNPs, a separate aqueous phase was prepared consisting of 25 μg of luciferase mRNA or GFP mRNA in 10 mM citrate buffer (pH 3). For co-delivered LNPs, a separate aqueous phase was prepared with 25 μg of total mRNA at a mass ratio of ABE to sgRNA specified in the text in 10 mM citrate buffer (pH 3). For screening LNPs, the ethanol and aqueous phases were combined at a 3:1 ratio through channels in a microfluidic device facilitated by a Pump33DS syringe pump (Harvard Apparatus, Holliston, MA). For co-delivered LNPs, the ethanol and aqueous phases were rapidly mixed and incubated for 15 minutes. All LNPs were dialyzed against 1X PBS for 2 hours before sterile filtration via 0.22 μM filters and storage at 4° C. All materials were prepared and handled ribonuclease-free throughout synthesis, formulation, and characterization steps.

### LNP Characterization

Zetasizer Nano (Malvern Instruments, Malvern, U.K.) was used to measure in triplicate the diameter (z-average) and polydispersity index (PDI) of LNPs suspended in 1×PBS. Zeta potential measurements were collected in triplicate using the Zetasizer Nano and DTS1070 zeta potential cuvettes (Malvern Panalytical, Malvern, U.K.) for each LNP diluted in deionized water.

pKₐ measurements were conducted as previously described. Buffered solutions of 150 mM sodium chloride, 20 mM sodium phosphate, 20 mM ammonium acetate, and 25 mM ammonium citrate were each adjusted to pH 2 to 12 in increments of 0.5. 200 μL of each pH-adjusted solution was combined with 5 μL of each LNP formulation in black 96-well plates in triplicate. TNS [6-(p-toluidinyl) naphthalene-2-sulfonic acid] was added to each well at a final TNS concentration of 6 μM. Fluorescence intensity was read on an Infinite 200 Pro plate reader (Tecan, Morrisville, NC), and pKₐ was calculated as the pH at which the fluorescence intensity was 50% of its maximum value, reflective of 50% protonation.

Encapsulation efficiencies were calculated using Quant-iT RiboGreen (Thermo Fisher Scientific, Waltham, MA) assay as previously described in the literature. Briefly, equal concentrations of LNPs were treated with either Triton X-100 (Sigma-Aldrich, St. Louis, MO) or left untreated, and after 10 minutes, the groups were plated in triplicate in black 96-well plates alongside RNA standards. The fluorescent RiboGreen reagent was added per manufacturer instructions, and the resulting fluorescence was measured on a plate reader. A standard curve was used to quantify RNA content and calculate encapsulation efficiency.

### Animal Experiments

BALB/c (stock #000651), B6.129 (Cg)-Gt (ROSA) 26Sor^{tm4(ACTB-tdTomato.-EGFP)Luo/J (R}26^{mTmG}, stock #007676), and B6.126S-Idua^{tm1.1Kmke}/J (called Idua-W392X, stock #017681) were purchased from The Jackson Laboratory (Bar Harbor, ME).

### In Utero, Neonatal, and Adult Mouse ICV Injection

In utero, neonatal, and adult ICV injection were performed as previously described in the literature. Time-dated pregnant female BALB/c mice at 18 days gestation were used to screen the LNP library in vivo. Briefly, under isoflurane anesthesia, a midline laparotomy was performed to expose the uterine horns. A dissecting microscope was used to identify the left lateral ventricle of each fetus. Either PBS or a total mRNA dose of 1 mg/kg of LNPs suspended in 4 μL was injected into the lateral ventricle using an 80-μm beveled glass micropipette and an automated microinjector (Narishige IM-400 Electric Microinjector, Narishige International USA Inc., Amityville, NY). After successful injection, confirmed by visualizing clearance of the injectate and transient swelling of the ventricle, the uterus was returned to the peritoneal cavity, the abdomen was closed with a single layer of 5-0 absorbable suture, and mice were allowed to recover in a warm cage.

P0 BALB/c or Idua-W392X pups were anesthetized on ice and then wiped with a cotton swab soaked in 70% ethanol to better visualize cranial landmarks. Injection sites were identified via a dissecting microscope at ⅖ of the distance from the lambda suture to each eye. Either PBS or a total mRNA dose of 1 mg/kg of LNPs suspended in 4 μL was injected into the lateral ventricles using an 80-μm beveled glass micropipette and an automated microinjector. After successful injection, confirmed by visualizing clearance of the injectate and transient swelling of the ventricle, pups were allowed to recover on a warming pad and returned to a nursing foster mother.

Pre-operatively, adult BALB/c mice were given oral meloxicam and then placed under isoflurane anesthesia. After administration of subcutaneous bupivacaine, a midline craniotomy was performed, subcutaneous and muscle tissue were separated, and the bregma and lambda suture areas were cleaned. A 31-gauge insulin syringe was used to gently drill through the skull into the target area (lateral ventricle), approximately 1 mm lateral and 0.3 mm anterior to the bregma, prior to injection of either PBS or a total mRNA dose of 1 mg/kg of LNPs suspended in 10 μL was injected into the lateral ventricles. For all parts of this procedure, the skull was kept moist with sterile PBS. Following injection, skin was sutured closed with running 5-0 absorbable suture, and animals were allowed to recover in a warm cage.

### Luciferase Imaging

Mice were imaged 4 hours after injection with LNPs on an in vivo imaging system (IVIS, PerkinElmer, Waltham, MA). Ten minutes before imaging, dams or neonates were injected intraperitoneally with D-luciferin (150 mg/kg) and potassium salt (Biotium, Fremont, CA). Anesthetized mice were then placed supine into the IVIS, and luminescence signal was detected with a standardized exposure time. For dams, a midline laparotomy was performed to expose the uterine horns, and luciferase imaging was repeated. After sacrifice, individual fetuses from each dam were individually imaged via IVIS. Finally, both fetuses and neonates were dissected, and the brain and liver were imaged by IVIS. Image analysis was conducted in the Living Image software (PerkinElmer). To quantify luminescent flux, a rectangular region of interest (ROI) was placed over each sample (fetus, neonate, organ of interest), and an ROI of the same size was placed in an area without any luminescent signal in the same image. Normalized flux was calculated by dividing total flux from the sample area by the total flux from the background area.

R26^{mT/mG} mouse studies and brain immunohistochemistry

P0 R26^{mT/mG} neonates were injected ICV with either PBS or C3 LNP. One week after injection, animals were harvested by standard transcardial perfusion using 4% PFA followed by overnight fixation. After 24 hours, samples were transferred to 30% sucrose for cryoprotection. Tissues were embedded in OCT compound and sectioned on a cryostat. Sections were mounted on Superfrost Plus slides (Thermo Fisher Scientific) and were frozen at −80° C.

For immunostaining, slides were dried at 60° C. via a slide warmer and rehydrated in 1×PBS. Tissues were blocked with 10% donkey serum (Sigma-Aldrich) followed by overnight incubation with 1:500 dilutions of the following primary antibodies: NeuN (Rabbit mAb #12943, Cell Signaling Technology, Danvers, MA), IBA1 (Rabbit mAb 019-19741, FUJIFILM Wako Pure Chemical Corporation, Richmond, VA), and GFAP (Rabbit mAb GA524, Agilent Technologies, Wilmington, DE). After 24 hours, slides were washed with 1×PBS and incubated with Alexa Fluor-conjugated secondary antibodies for 2 hours at room temperature. Slides were mounted on Fluoroshield Mounting Medium with DAPI (Abcam, Cambridge, U.K.) and were imaged on a fluorescence microscope (BZ—X, Keyence, Osaka, Japan).

### In Vitro LNP Optimization Studies

Neuro-2a cells (ATCC no. CCL-131) were cultured in Dulbecco's Modified Eagle's Medium (DMEM) with l-glutamine (Thermo Fisher Scientific) supplemented with 10% fetal bovine serum (FBS) (Gibco, Dublin, Ireland) and 1% penicillin-streptomycin (Gibco). For the DOE LNP screen and N:P ratio LNP screen, cells were plated in a tissue culture treated 96 well plate at a cellular density of 1000 cells/μL, incubated overnight, and dosed with LNPs encapsulating GFP mRNA at a total mRNA concentration of 100 ng. After 24 hours, cells were detached via trypsin, washed with 1×PBS, and resuspended in flow cytometry (FACS) buffer (Ca²⁺/Mg²⁺ Free PBS, 0.5% BSA, 0.5 mM EDTA). Samples were analyzed for fluorescence (GFP+) via flow cytometry (BD FACSAria™ Cell Sorter, Haryana, India). Viability was assessed via Live/Dead™ Cytotoxicity Kit (Thermo Fisher Scientific).

For the LNP ABE to sgRNA ratio screen, primary fibroblasts or primary neurons were harvested from a single adult Idua-W392X mouse. The mouse was euthanized and either lung tissue or brain tissue was isolated. These tissues were digested mechanically and filtered through 100 μm cell strainers. Cells were washed with 1×PBS and subsequently cultured in DMEM supplemented with 15% FBS and 1% penicillin-streptomycin. Either primary fibroblasts or primary neurons were plated in a tissue culture treated 96-well plate at a cellular density of 1000 cells/μL, incubated overnight, and treated with LNPs at a range of ABE to sgRNA ratios or a dose range. After 5 days, cells were detached via trypsin, washed in 1×PBS, and resuspended in QuickExtract™ DNA Extraction Solution (Lucigen Corporation, Middleton, WI). Q5 polymerase was used to amplify the genomic region encompassing the W392X mutation with the following primers (F: 5′-TGCTAGGTATGAGAGAGCCA-3′ (SEQ ID NO:3) and R: 5′-AGTGTAGATGAGGACTGTGGT-3′ (SEQ ID NO:4)) at an annealing temperature of 66° C. PCR products were evaluated using a 1% agarose gel, purified using the Qiagen PCR Purification Kit as per manufacturer's recommendations, and analyzed by Sanger sequencing (Azenta Life Sciences, Chelmsford, MA).

Idua-W392X mouse studies (on- and off-target editing, IDUA activity, tissue GAG assay)

P0 Idua-W392X neonates were injected ICV with C3.MPS LNPs at a total mRNA dose of 1 mg/kg. Untreated age- and sex-matched Idua-W392X mice and wild-type B6 mice served as positive and negative controls, respectively. One month after injection, animals were sacrificed, and tissue from three separate regions of the brain (i.e., forebrain, midbrain, and hippocampus), liver, and gonads was harvested. DNA was extracted from each tissue using the Qiagen DNEasy Blood and Tissue Kit according to manufacturer's instructions (Qiagen, Hilden, Germany), PCR amplified and purified as described elsewhere herein, and analyzed via next-generation sequencing (Azenta Life Sciences). The top ten-off target sites were predicted using CRISPOR and ranked using the Cutting Frequency Determination off-target score. These sites were PCR amplified using Platinum SuperFi II Hi-Fidelity DNA Polymerase (Thermo Fisher Scientific), PCR amplified and purified (described above), and analyzed via next-generation sequencing (Azenta Life Sciences).

Tissue IDUA activity was assayed as previously described in the literature. After tissue harvest, 20 mg samples were homogenized with 0.1% Triton X-100 lysis buffer using a TissueLyser LT (Qiagen). IDUA enzyme activity was induced in tissue homogenate using 4-methyl-umbelliferyl-α-L-iduronide (Glycosynth, Warrington, U.K.). After incubation, reactions were arrested using glycine carbonate buffer, and fluorescence was measured via plate reader. A standard curve was generated with 4-methylumbelliferone (Sigma-Aldrich) suspended in arrestant buffer at a detection sensitivity of 80. Enzyme activity was normalized to tissue homogenate protein content measured via Pierce BCA Protein Assay Kit according to manufacturer's instructions (Thermo Fisher Scientific). After obtaining IDUA enzyme activity, 0.5 mL of papain solution (Sigma-Aldrich) was added to homogenized tissue lysates. Samples were incubated at 65° C. for 3 hours and then centrifuged to clarify supernatant. Total sulfated GAG content in each sample was assessed using the Blyscan Glycosaminoglycan Assay (Biocolour, Carrickfergus, U.K.) according to manufacturer's instructions. Tissue GAG content was normalized to tissue lysate protein content.

### Idua-W392X Mouse Studies (Cytokine Analysis, Anti-PEG IgM Antibody)

A separate cohort of P0 Idua-W392X were injected ICV with either PBS, C3.MPS, or MC3.MPS LNPs at a total mRNA dose of 1 mg/kg. Animals were terminally bled at 24 hours prior to serum cytokine analysis using a 25-proinflammatory cytokine panel (MilliporeSigma, Burlington, MA) according to manufacturer's instructions. A standard curve for each plate was prepared by serial dilution of the provided standards in the appropriate dilutant. Multiplex plates were run on a MAGPIX® system (Luminex Corporation, Austin, TX) with a minimum of 50 beads analyzed per region. Each cytokine was assessed using a five-parameter regression algorithm and normalized to the protein concentration in the sample as determined by microBCA assay.

A separate cohort of P0 Idua-W392X neonates and adults were injected ICV with either PBS or C3.MPS at a total mRNA dose of 1 mg/kg. Animals were terminally bled after 7 days, and the concentration of circulating IgM anti-PEG antibodies was assessed by Mouse Anti-PEG IgM ELISA (Life Diagnostics Incorporated, Chester, PA) as per manufacturer's instructions. The concentration of IgM anti-PEG antibody was calculated based on a standard curve.

### LNP Administration to Fetal Macaque and Brain Immunohistochemistry

A cynomolgus macaque (Macaca fasicularis) was used for a proof-of-principle non-human primate (NHP) study. Macaques underwent timed mating with pregnancy confirmed through ultrasound evidence of a fetal pole and fetal heart activity. Dating was performed as previously reported, and in utero ICV injection was performed at 0.61G. In brief, anesthesia was induced via sevoflurane. A 25G Quincke needle (Becton-Dickenson, Franklin Lakes, NJ) was used to target the lateral ventricle closest to the anterior maternal abdomen under continuous ultrasound imaging. C3 LNP was injected as a slow bolus, and transient ventricular swelling validated successful delivery. The needle was removed, and both mother and fetus were monitored post-operatively. Animals were sacrificed after 48 hours, and macaque brains were harvested using isoflurane and pentobarbitone, cardiac puncture, and perfusion of 1% PFA. After 24 hours, samples were transferred to 30% sucrose for cryoprotection, embedded in OCT compound, sectioned on a cryostat, and mounted on Superfrost Plus slides (Thermo Fisher Scientific). For immunostaining, slides were dried at 60° C. via a slide warmer and rehydrated in 1X PBS. Tissues were blocked with 10% donkey serum (Sigma-Aldrich) and incubated overnight with 1:100 dilution of GFP antibody (Rabbit mAb ab290). After 24 hours, slides were washed with 1×PBS and incubated with an Alexa Fluor-conjugated secondary antibody for 2 hours at room temperature. Slides were mounted on Fluoroshield Mounting Medium with DAPI and were imaged on a fluorescence microscope.

### LNP Stability in Human Fluids

LNP stability in patient-derived fluids was assessed utilizing a previously published protocol. Briefly, C3.MPS LNPs were incubated in a range of human serum or CSF percentages −0%, 25%, 50%, 75%, and 100% (v/v) for 30 minutes at 37° C. under gentle agitation at 300 rpm. After 30 minutes, each sample was diluted 100× in 1×PBS and transferred to a standard cuvette for size and PDI measurement via Zetasizer Nano. Additional samples were prepared via the same protocol, diluted in deionized water, and transferred to a DTS1070 zeta potential cuvette for zeta potential measurement via Zetasizer Nano.

### Isolation of Patient-Derived Precision Cut Brain Slices and Brain Cells

Brain tissue was carefully dissected with only minimal use of bipolar forceps to ensure tissue integrity and directly transferred into ice-cold patient cerebrospinal fluid, equilibrated with carbogen (95% 02, 5% C₀2). In the laboratory, tissue was trimmed and 300 μm slices were prepared using a vibratome (Leica Biosystems, Wetzlar, Germany) in the same oxygenated and cooled CSF. The slices were transferred to 12-well plates and cultured in primary brain media modified from prior protocols: 50% DMEM, 48% Neurobasal™ Media (Thermo Fisher Scientific), 1× GlutaMAX™ (Gibco), and 20 mM HEPES (Thermo Fisher Scientific).

Primary human brain cells were isolated from brain tissue via an established protocol. Patient-derived brain tissue, as described elsewhere herein, was mechanically dissociated before enzymatic digestion in papain and DNase I (Invitrogen Corporation, Waltham, MA) for 30 minutes at 37° C. The cell suspension was passed through a 100 μm cell strainer, centrifuged, and resuspended in primary brain media. Cells were plated onto poly-d-lysine-coated cell culture surfaces, and 50% of culture media was exchanged every 48 hours.

### LNP Studies in Patient-Derived Cells and Tissue

Patient-derived primary cells were seeded at a density of 10,000 cells in a poly-d-lysine coated 96-well plate and allowed to incubate overnight. Cells were subsequently treated with either PBS or C3 LNPs encapsulating GFP at a total mRNA concentration of 100 ng. After 24 hours, cells were detached via light cell scraping, washed with 1×PBS, and resuspended in FACS buffer. Samples were analyzed for fluorescence (GFP+) via flow cytometry. Viability was assessed via Live/Dead™ Cytotoxicity Kit (Thermo Fisher Scientific).

On the day of treatment, precision cut brain slices were cultured in 50% primary brain media and 50% human CSF. Slices were treated with either PBS or C3.MPSmut LNPs at a total mRNA dose of 500 ng, and 24 hours later, the culture solution was switched back to primary brain media. After 5 days, slices were mechanically digested, passed through a 100 μm cell strainer, and resuspended in QuickExtract™ DNA extraction solution. The genomic region encompassing the human W402X mutation was amplified with KAPA HiFi DNA Polymerase (Roche, Basel, Switzerland) using the following primers (F: 5′-CAATGCCTTCCTGAGCTACCAC-3′ (SEQ ID NO:5), R: 5′-AGGTAGCGCGTGACGTAGAC-3′ (SEQ ID NO:6)) at an annealing temperature of 57° C. PCR products were evaluated using a 1% agarose gel, purified using the Qiagen PCR Purification Kit as per manufacturer's recommendations, and analyzed by Sanger sequencing (Azenta Life Sciences).

### Statistical Analysis

For the LNP library screen, all luciferase quantifications represent the mean and standard error of the mean (SEM) acquired from at least three animals per treatment group. The means were compared by one-way ANOVA with post hoc Dunnett's test. For LNP in vitro optimization, all values represent the mean and SEM of experiments executed in triplicate. Means were compared by one-way ANOVA with post-hoc Dunnett's test. For the Idua-W392X experimental cohort, all values represent the mean and SEM acquired from a minimum of four animals per group. IDUA activity and GAG content were compared by two-way ANOVA across both brain region and treatment group with post hoc Šídák's multiple comparisons test. For cytokine analysis, all values represent the mean and SEM from a minimum of three animals per treatment groups. Cytokine data was compared two-way ANOVA across both specific cytokine and treatment group with post hoc Šídák's multiple comparisons test. Anti-PEG IgM antibody levels were collected in triplicate were compared via Student's t test with a=0.05. LNP studies in patient-derived fluids, cells, and tissues were completed in triplicate with values reported as mean and SEM. For all experiments, outliers were detected using Grubbs' test and removed from analysis.

### Example 1: Synthesis and characterization of an ionizable LNP library

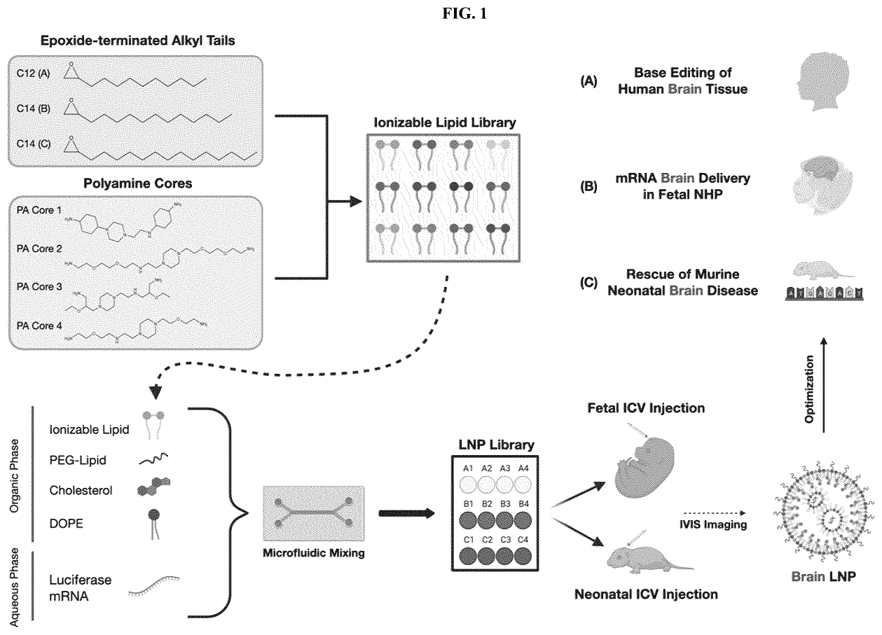

A library of 12 LNPs was prepared according to procedures known to those of ordinary skill in the art. First, ionizable lipids were synthesized by nucleophilic addition, whereby alkyl tails denoted by tail length (i.e., A=C₁₂, B═C₁₄, C═C₁₆) were reacted with polyamine molecules labeled numerically (i.e., 1 through 4) to form polyamine-lipid cores. These ionizable lipids were combined with cholesterol, 1,2-dioleoyl-sn-glycero-3-phosphoethanolamine (DOPE), and lipid-anchored PEG at ratios determined by previous optimization studies in adult mice and then mixed with firefly luciferase mRNA through a herringbone-style microfluidic device (FIG. 1).

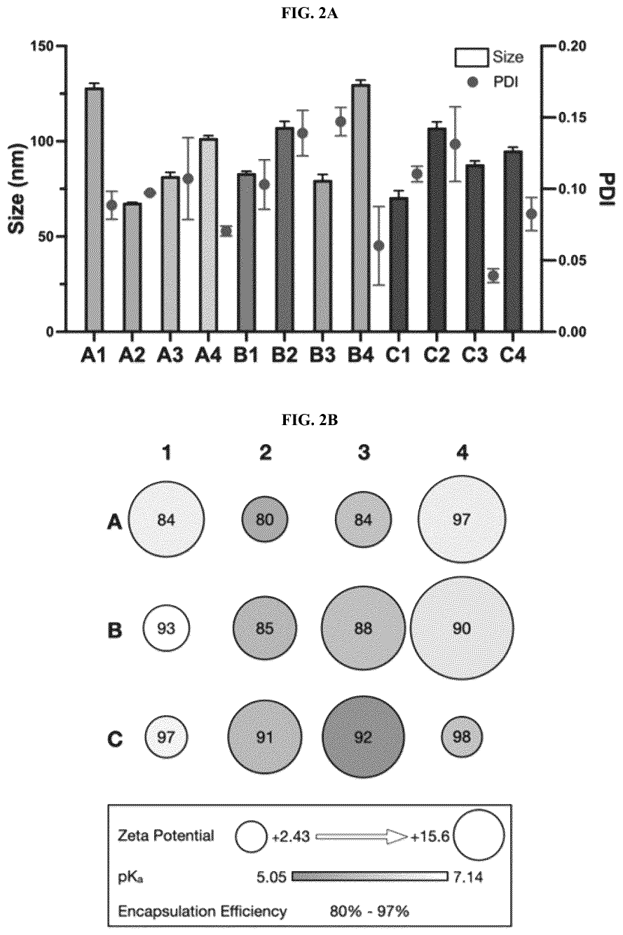

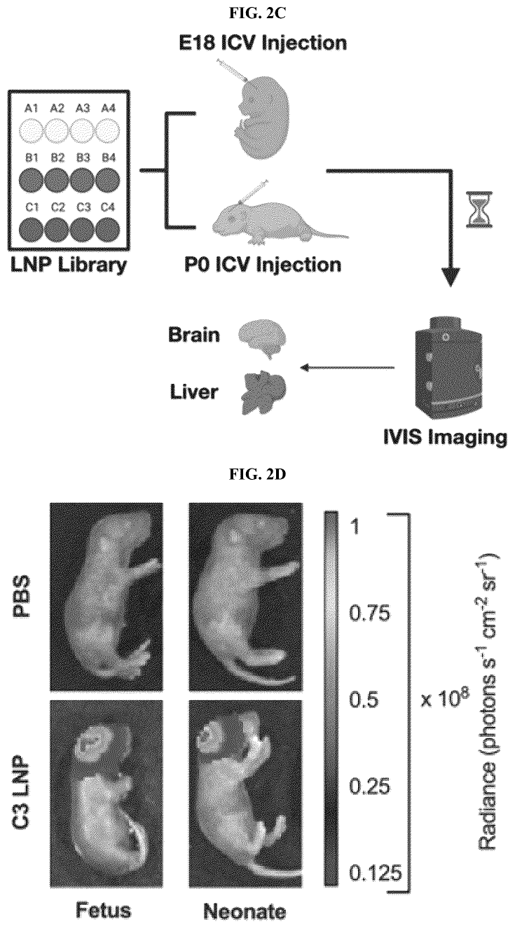

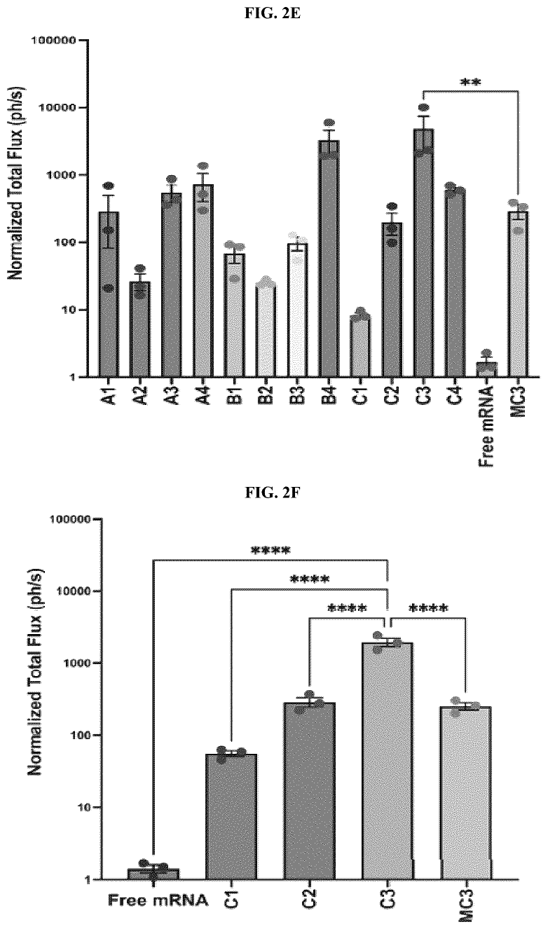

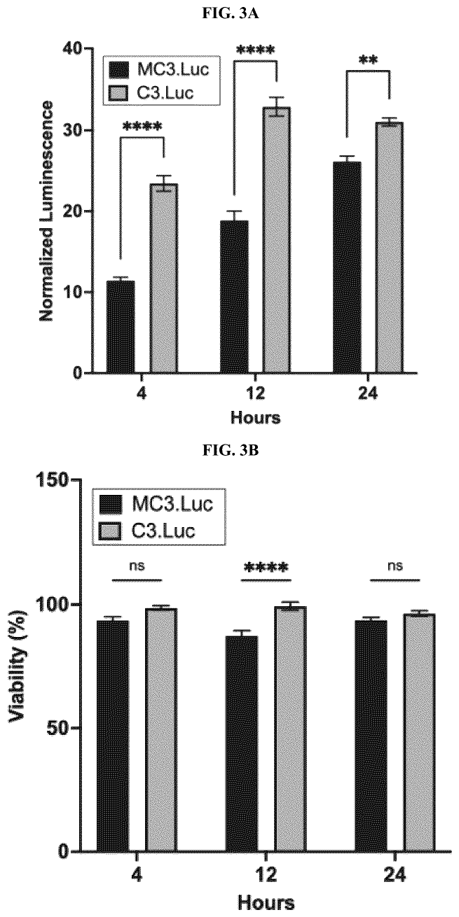

The resultant library of LNPs was characterized by size, encapsulation efficiency, pKₐ, and zeta potential. Hydrodynamic diameter for all LNP formulations, as measured via dynamic light scattering intensity, ranged from 67.8 to 128.3 with a maximum polydispersity value of 0.154 (FIG. 2A), indicating small and monodisperse LNPs. The ability for LNPs to encapsulate mRNA was evaluated via fluorometric RiboGreen assay. Encapsulation efficiencies were high, ranging from 79.9% to 97.6% (FIG. 2B). Next, LNPs were assessed for their pKₐ, which reflects their ability to undergo endosomal escape and release nucleic acid cargo. All LNP formulations had a pKₐ value between 5.05 and 7.14 (FIG. 2B), which is within an acceptable range for in vivo nucleic acid delivery. Finally, the zeta potential, a function of the surface charge, was measured for each LNP, since charge modulates both the protein corona adherent to the surface of LNPs and ionic interactions with cell membranes. In this library, LNP zeta potentials ranged from 15.6 mV to 2.43 mV (FIG. 2B). Overall, the physiochemical properties of these LNPs were similar to those measured for a structurally related LNP library screened for delivery to the fetal liver (Table 1).

TABLE 1
Selected physiochemical properties of
exemplary LNPs

| | | | | | Zeta |
|---|---|---|---|---|---|
| | Size | | EE | | Potential |
| LNP | (nm) | PDI^{a} | (%)^{b} | pKₐ | (mv) |
| A1 | 128 | 0.094 | 84.2 | 6.81 | 8.43 |
| A2 | 68 | 0.103 | 79.9 | 5.57 | 2.99 |
| A3 | 82 | 0.113 | 83.5 | 5.97 | 4.55 |
| A4 | 102 | 0.075 | 97.0 | 6.76 | 11.1 |
| B1 | 83 | 0.109 | 93.4 | 7.14 | 3.12 |
| B2 | 107 | 0.146 | 85.1 | 5.79 | 5.89 |
| B3 | 80 | 0.154 | 88.1 | 5.94 | 10.4 |
| B4 | 130 | 0.064 | 90.2 | 6.60 | 15.6 |
| C1 | 71 | 0.116 | 96.6 | 6.87 | 2.57 |
| C2 | 107 | 0.138 | 91.1 | 5.83 | 7.89 |
| C3 | 88 | 0.043 | 92.0 | 5.05 | 9.78 |
| C4 | 95 | 0.087 | 97.6 | 6.01 | 2.43 |

^{a}Polydispersity index;
^{b}encapsulation efficiency.

### Example 2: LNP-mediated mRNA delivery to the perinatal mouse brain

After characterizing the LNP library, the library was evaluated for mRNA delivery to the brain in fetal and neonatal BALB/c mice (FIG. 2C). First, LNPs encapsulating luciferase mRNA were injected ICV into the lateral ventricles of gestational day (E) 18 BALB/c fetuses. This gestational age was chosen to characterize the ability of the LNP library to deliver mRNA to the brain in utero at a neural developmental stage akin to that of a mid-gestation human fetus, which represents a timepoint where therapeutic intervention is technically feasible. Furthermore, ICV injection was selected as the mode of introduction since it has been used clinically to safely and effectively deliver therapeutic agents for a broad range of diseases. Each mouse fetus has its own gestational sac, permitting injection of multiple fetuses in a single dam without crossover of injectate. At least two fetuses in each pregnancy were injected with phosphate-buffered saline (PBS) as an internal negative control. Furthermore, naked luciferase mRNA was included as a treatment group to demonstrate baseline nucleic acid uptake in the brain without a delivery carrier. Finally, DLin-MC3-DMA ionizable lipid (i.e., an FDA approved industry standard) was used to encapsulate luciferase mRNA as a positive control.

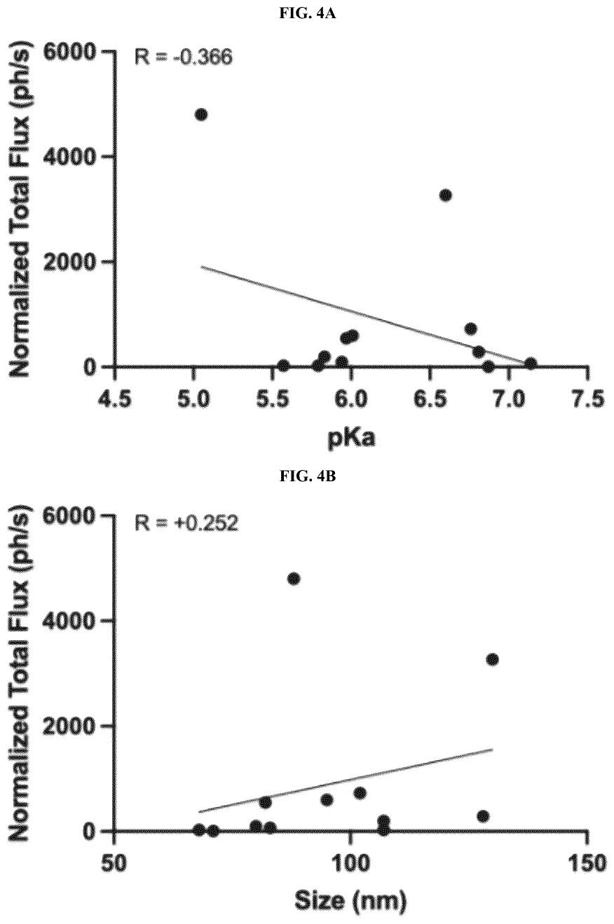

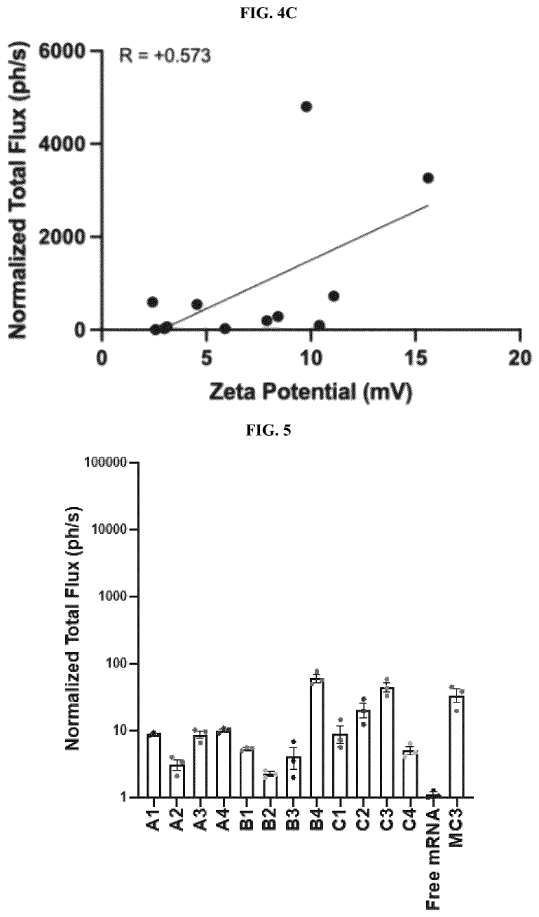

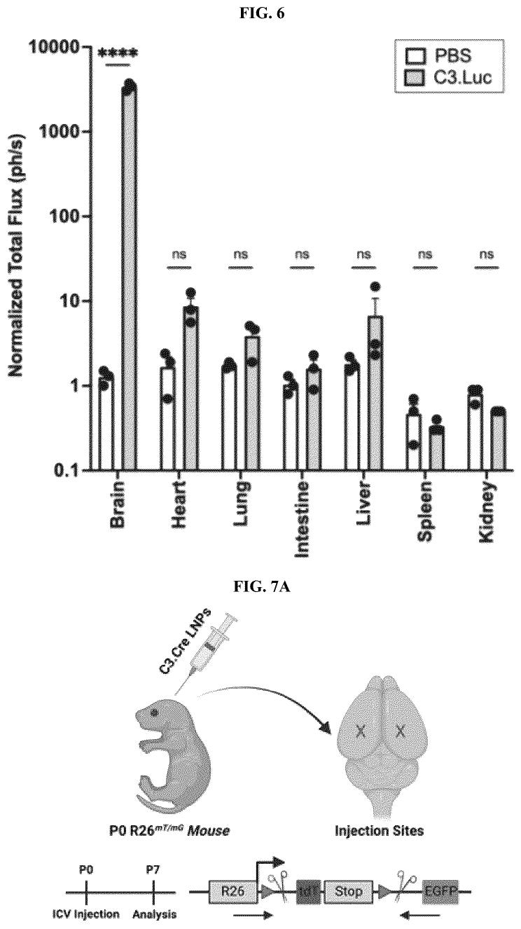

Fetuses were assessed after 4 h after ICV injection for luciferase expression using an in vivo imaging system (IVIS). This time point was selected on the basis of previous studies of in vitro verification of LNP-mediate luciferase expression by 4 h (FIGS. 2E-2F). Direct visualization of transfection efficiency using an in vivo imaging system (IVIS) in fetuses injected with the LNP library (FIG. 2D) demonstrated a wide range of functional mRNA delivery to the fetal brain. All LNP formulations yielded greater mRNA delivery than injection of naked mRNA, confirming that LNP encapsulation facilitates intracellular delivery, while each ionizable lipid had variable performance, demonstrating the crucial role of the ionizable lipid within the LNP in mRNA delivery (FIG. 2E). Further, trends for in vivo transfection efficacy were observed in the fetal brain with decreasing pKₐ, increasing size, and increasing zeta potential (FIGS. 4A-4C). While several LNP formulations demonstrated equivalent mRNA delivery to the fetal brain relative to DLin-MC3-DMA LNPs, the top-performing LNP formulation (i.e., C3 LNP) resulted in 17-fold greater luciferase expression compared to this industry standard (p<0.05). Of note, there was minimal expression of luciferase observed in the liver with all the LNPs in this library (FIG. 5), demonstrating the localized nature of both the mode of introduction and the delivery platform. Additionally, strong luciferase expression was observed in the fetal brain but no significant expression in the heart, lung, intestine, liver, spleen, or kidney relative to a PBS-injected control (FIG. 6).

In this in vivo screen, long-term survival post-injection was not assessed as animals were sacrificed within hours of treatment. However, fetal mouse ICV injection has previously been associated with intrauterine mortality due to the technical challenge of this procedure in small animals. In contrast, postnatal day 0 (P0) mouse ICV injections are technically easier, associated with good long-term survival, and still approximate the CNS development of a mid-gestation human fetus. Therefore, additional studies were performed to determine if LNP-mediated mRNA delivery to the fetal brain was maintained in a neonatal mouse model supporting the use of this model for future studies.

High-, mid-, and low-performing LNPs were selected from the previous LNP library and injected ICV into P0 BALB/c neonates. IVIS imaging of neonatal BALB/c brains confirmed that C3 LNPs had significantly greater mRNA delivery than C2 LNPs (p<0.05) and C1 LNPs (p<0.05), replicating the trend observed in the fetal mouse model (FIG. 2F).

### Example 3: C3 LNPs facilitate gene modulation in cells of the murine brain ventricular lining

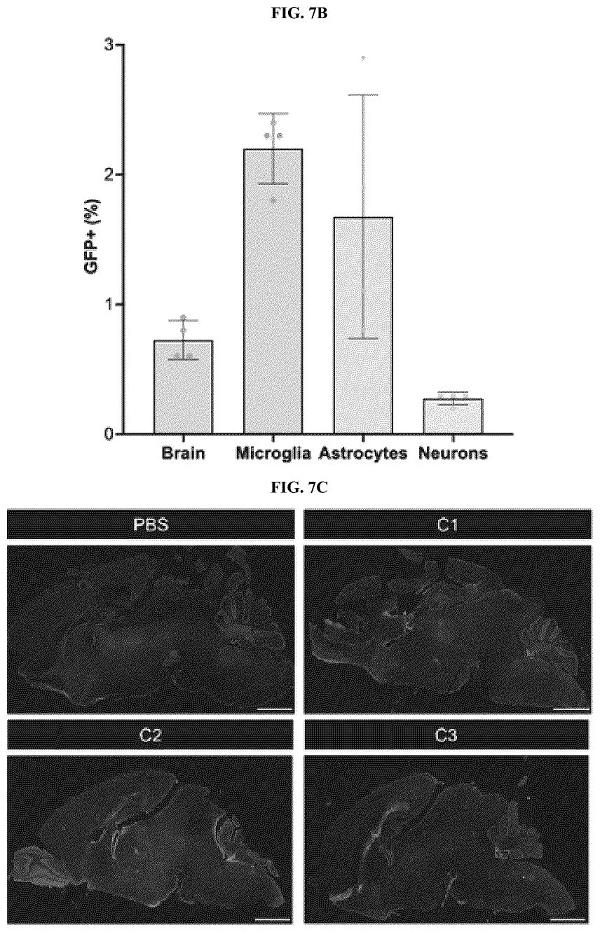

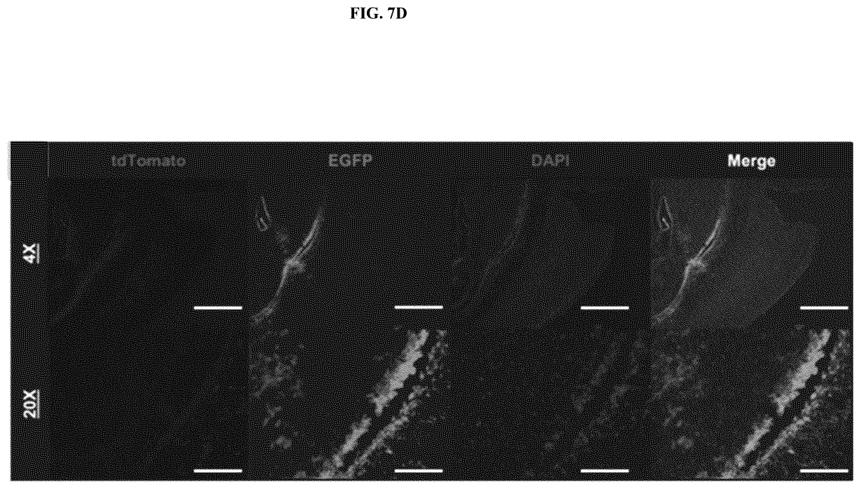

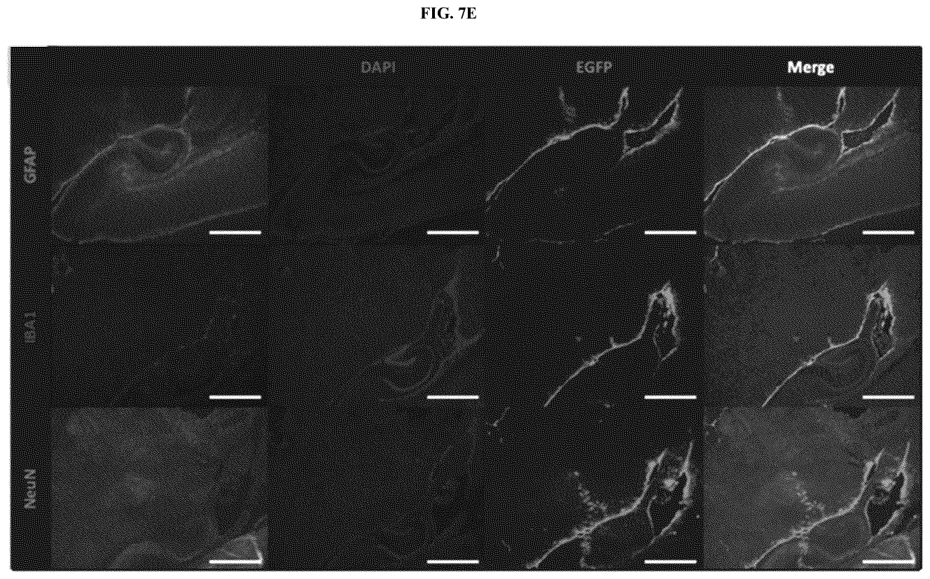

To gain insight into the cellular tropism of C3 LNP-mediated mRNA delivery in the neonatal brain, C3 LNPs encapsulating Cre recombinase mRNA were injected ICV at a dose of 1 mg/kg in P0 R26^{mT/mG} mice. R26^{mT/mG} mice possess loxP sites on either side of a membrane-targeted tdTomato (mT) cassette and constitutively express strong red fluorescence in all tissues (FIG. 7A). Upon expression of translated Cre protein, the mT cassette cDNA is deleted along with a transcriptional stop cassette, resulting in expression of a downstream mG-enhanced green fluorescent protein (mG-EGFP) cassette. Thus, successful LNP-mediated delivery of Cre mRNA leads to expression of green fluorescence in the transfected cell.

One week after injection, brain tissue was analyzed for gene modulation via flow cytometry and histology. Low level GFP expression was noted in the brain with higher expression in microglia and astrocytes compared to neurons (FIG. 7B). Whole brain histology confirmed that GFP expression was localized to areas adjacent to ICV injection, with limited diffusion to internal structures (FIGS. 7C-7D). Cell-specific immunohistochemistry revealed strong transfection of microglia lining the ventricular space and in nearby astrocytes and neurons (FIG. 7E). Despite low whole brain gene modulation, transfection of cells in close proximity to or lining the ventricles may be beneficial for production of therapeutic proteins to be secreted into the CSF.

### Example 4: In Vitro Optimization and Validation of C3 LNPs for Co-Delivery of Base Editing Technology

The molar ratios at which the components of LNPs are combined can be optimized for specific applications to better encapsulate nucleic acid cargo, enhance uptake by target cells, or alter biodistribution and protein corona formation. In the initial library screen, LNPs were formulated with standard formulation parameters known to facilitate mRNA delivery. However, the optimal parameters to produce C3 LNPs specifically for base editing applications in the brain were unknown. Thus, a multi-stage optimization protocol of C3 LNPs was used, wherein the effects of excipient molar ratio, N:P ratio, and ratio of co-delivered mRNA on delivery performance in neural-origin cell lines were probed (FIG. 8A).

While the ionizable lipid, in this case C3, is a major determinant of intracellular delivery by both directly complexing with mRNA and facilitating endosomal escape, the remaining excipients also play critical roles: cholesterol provides stability and enables membrane fusion, DOPE supplies structural support, and PEG reduces aggregation and non-specific endocytosis. It has been previously demonstrated that modulating excipient molar ratios improves in vitro delivery to fetal lung cells and corresponds to enhanced in vivo performance following intra-amniotic LNP injection.

Herein was used orthogonal design of experiments (DOE) methodology to efficiently investigate a design space of 27 potential LNP formulations, generated using three molar ratios each of C3 ionizable lipid, cholesterol, and DOPE with a fixed PEG-lipid ratio (FIG. 8B). A representative library of 9 LNPs encapsulating GFP mRNA was screened for delivery and cytotoxicity in Neuro-2a cells (i.e., immortalized mouse neuroblasts isolated from brain tissue).

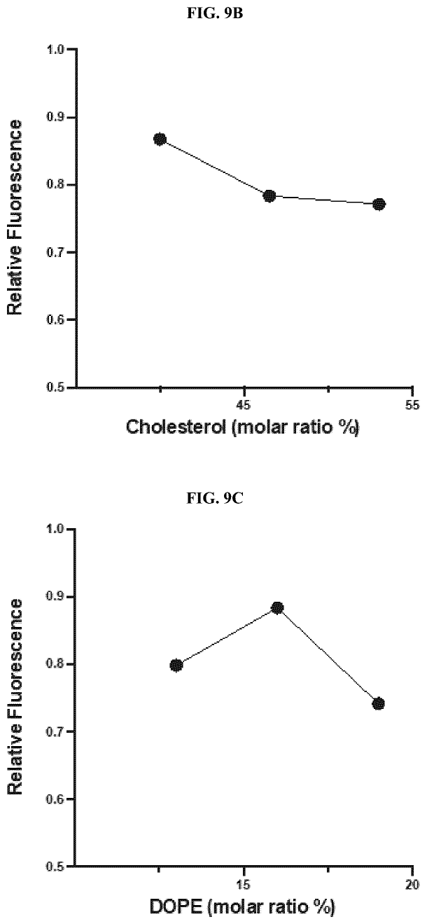

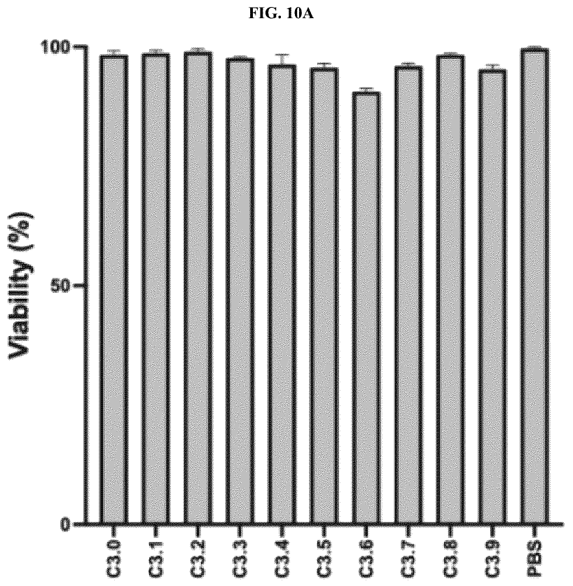

Although none of these formulations significantly enhanced mRNA delivery compared to the original C3 LNP formulation (FIG. 8C), relationships between the molar ratio of each excipient and mRNA delivery were identified. Specifically, improved mRNA delivery was observed with high molar ratios of ionizable lipid, low molar ratios of cholesterol, and moderate ratios of DOPE (FIGS. 9A-9C). Of note, these trends are distinct from those observed in prior studies that utilized different ionizable lipids, demonstrating the importance of exploring these parameters.

The N:P ratio is the quantitative relationship between nitrogen on the ionizable lipid to phosphate on mRNA. Previous studies have demonstrated that the N:P ratio impacts both nucleic acid delivery and toxicity with higher ratios leading to diminishing returns in cellular transfection due to increased cell death. The ideal N:P ratio is known to be distinct for each ionizable lipid. As such, C3 LNPs were formulated at a range of N:P ratios to encapsulate GFP mRNA and screened for delivery and cytotoxicity in Neuro-2a cells.

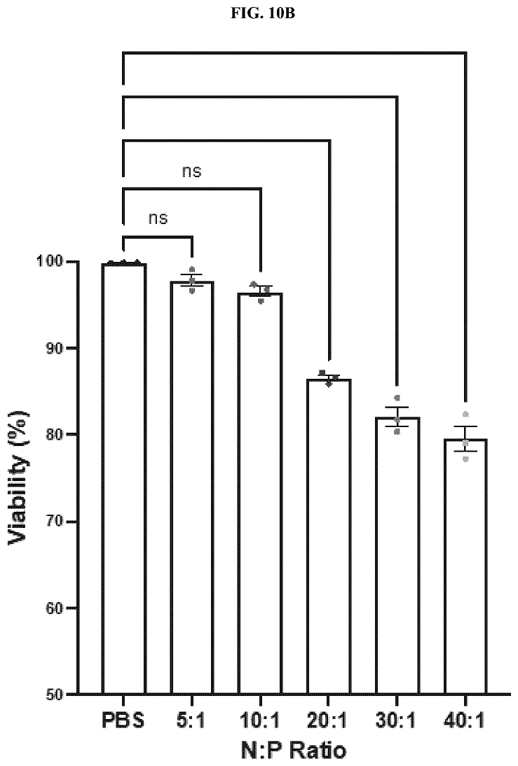

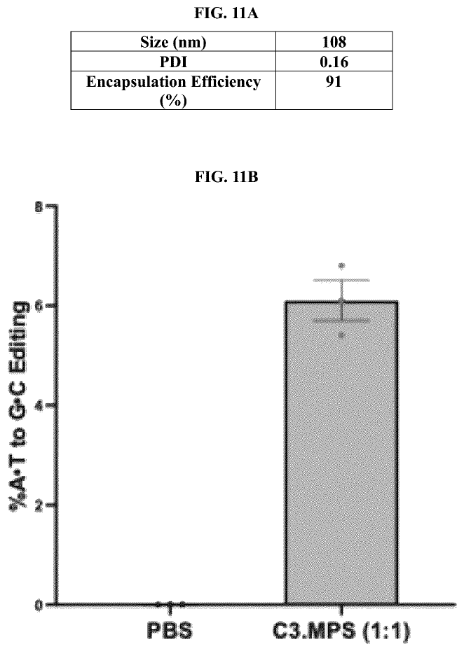

In comparison to the original N:P ratio of 10:1, LNPs formulated at an N:P ratio of 20:1 had a significant improvement in mRNA delivery (FIG. 8D, p<0.05) albeit with enhanced toxicity (FIGS. 10A-10B). To explore if this improvement was maintained in vivo, C3 LNPs were formulated with luciferase mRNA, for case of functional read-out, at both 10:1 and 20:1 N:P ratios and injected ICV in P0 BALB/c mice. IVIS imaging revealed that while both LNP formulations facilitated 2000-fold greater brain mRNA delivery relative to naked mRNA, there was no significant difference between the LNPs formulated at different N:P ratios (FIG. 8E). An N:P ratio of 10:1 was selected for further study to minimize the presence of potentially reactive, excess ionizable lipid in solution.

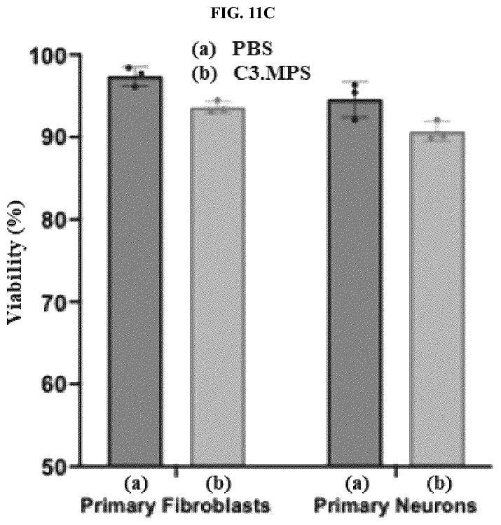

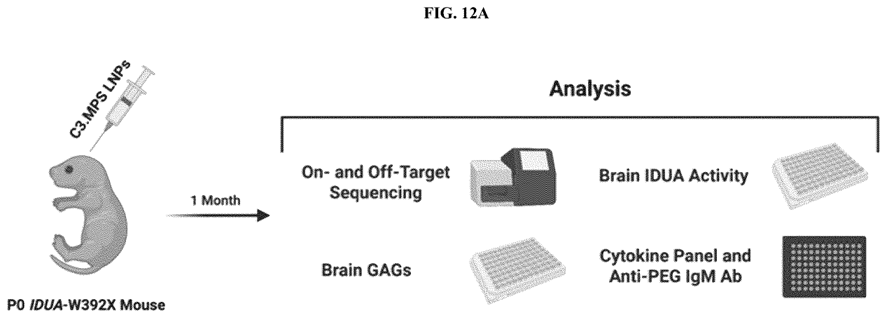

While reporter mRNA was used in the first two phases of in vitro optimization to improve throughput, for LNPs to be utilized for base editing applications, co-delivery of a base editor and sgRNA is necessary. Since few studies have demonstrated LNP-mediated base editing, the feasibility of LNP co-encapsulation of both a base editor and sgRNA was first evaluated. C3 LNPs were prepared using an adenine base editor (ABE) and an sgRNA specific for the Idua G→A (W392X) mutation present in the mouse model of MPS-IH at an RNA mass ratio of 1:1. Characterization of these LNPs showed a small size, albeit larger than C3 LNPs prepared with reporter mRNA, and high encapsulation efficiency (FIG. 11A). These LNPs were evaluated in primary fibroblasts derived from the Idua-W392X mouse and demonstrated low-level on-target base editing (FIG. 11B) with limited toxicity (FIG. 11C). To further optimize C3 LNPs, the effect of the mass ratio of ABE to sgRNA during LNP formulation on base editing efficiency was evaluated. Treatment of primary MPS-IH mouse fibroblasts revealed that C3 LNPs formulated at a 3:1 mass ratio of ABE to sgRNA (C3.MPS LNPs) resulted in significantly greater correction at the mutation site than the original formulation (FIG. 8F, p<0.05). Finally, C3.MPS LNPs were used to treat primary neurons derived from the MPS-IH mouse brain. Sequencing of LNP-treated cells showed dose-dependent base editing (FIG. 8G) with limited toxicity (FIG. 11C).

### Example 5: C3.MPS LNPs Mediate Biochemical Rescue of Disease in the Neonatal MPS-IH Mouse Brain

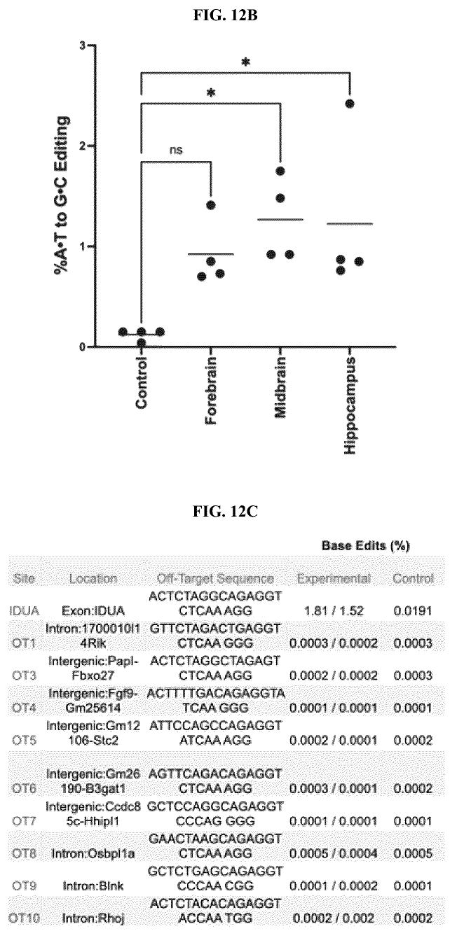

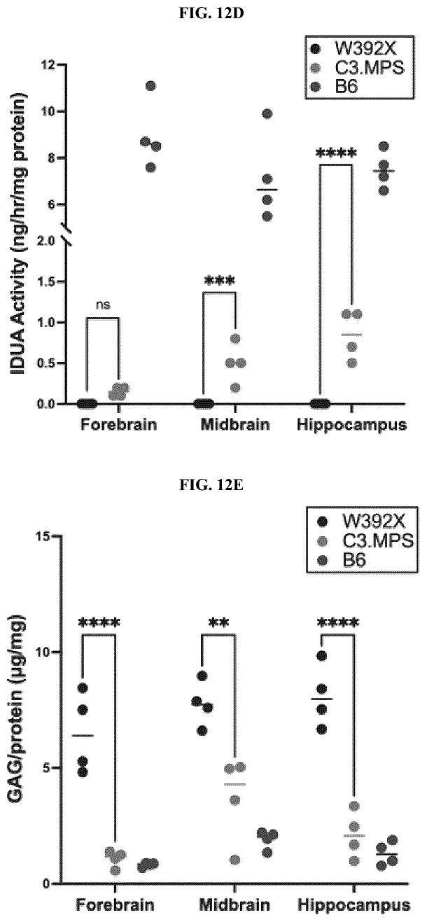

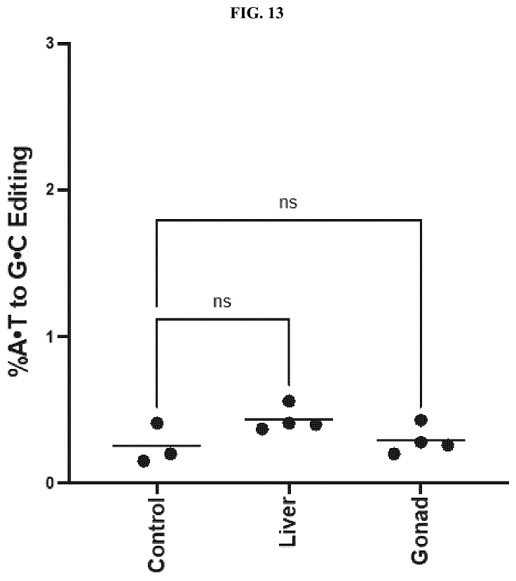

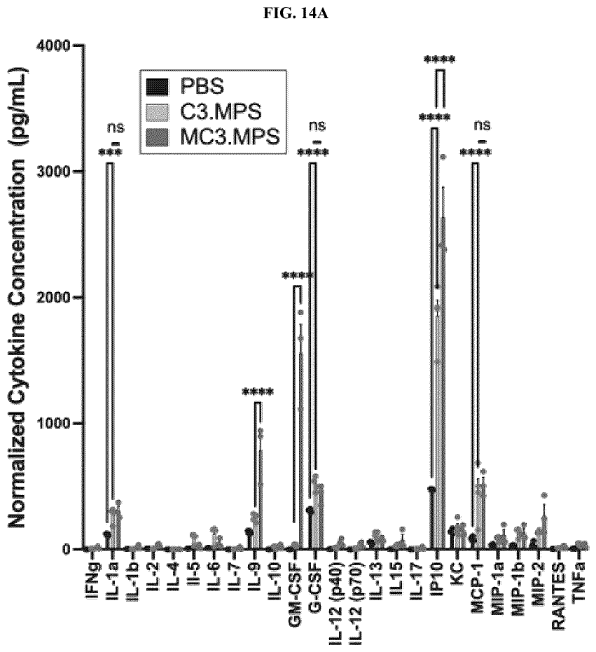

Following in vitro optimization, the optimized LNP formulation was tested in vivo in the Idua-W392X mouse model. An experimental cohort of P0 Idua-W392X neonates was injected ICV with C3.MPS LNPs at a dose of 1 mg/kg and harvested one month later for downstream genetic and biochemical analyses (FIG. 12A). PBS injected age- and sex-matched Idua-W392X mice and B6 mice served as controls. Survival after neonatal ICV injection was 100% in all experimental animals. Next-generation sequencing at one month demonstrated low-level on-target base editing at the disease-causing locus in several regions of harvested brain tissue (FIG. 12B). Overall editing efficiency in the brain was comparable to that observed previously in the R26^{mT/mG} mouse model. Notably, no editing above baseline was noted in genomic DNA isolated from the liver and gonads (FIG. 13), demonstrating similar brain specific delivery observed in the initial LNP library screen.

To characterize the genomic safety of this base editing strategy for murine MPS-IH delivered via C3.MPS LNPs, a published computational model was used to predict the most likely off-target base editing sites. At the 9 sites analyzed, no base edits above baseline were detected in samples from the experimental cohort of mice (FIG. 12C). This recapitulates the results of a prior off-target analysis, using the same ABE and sgRNA, delivered via adeno-associated virus.

Next, the existence of durable biochemical corrections in the brains of Idua-W392X mice treated with C3.MPS LNPs was explored. Like humans with MPS-IH, Idua-W392X mice have undetectable α-L-iduronidase (IDUA) enzyme activity and elevated tissue glycosaminoglycans (GAGs). At one month, mice treated with C3.MPS LNPs as neonates demonstrated increased IDUA activity in the midbrain (p<0.001, 6.97% of normal) and hippocampus (p<0.0001, 11.3% of normal) but not in the forebrain relative to untreated Idua-W392X mice (FIG. 12D). C3.MPS LNP-treated mice also had a significant reduction in GAGs, as measured by colorimetric assay, in the forebrain (p<0.0001), midbrain (p<0.01), and hippocampus (p<0.0001) (FIG. 12E). These data suggest that C3.MPS LNPs facilitate partial biochemical correction of disease in the neonatal Idua-W392X mouse brain, demonstrating potential for therapeutic rescue of brain disease in MPS-IH.

### Example 6: C3.MPS LNPs Produce Minimal Immunologic Response in the Neonatal MPS-IH Mouse

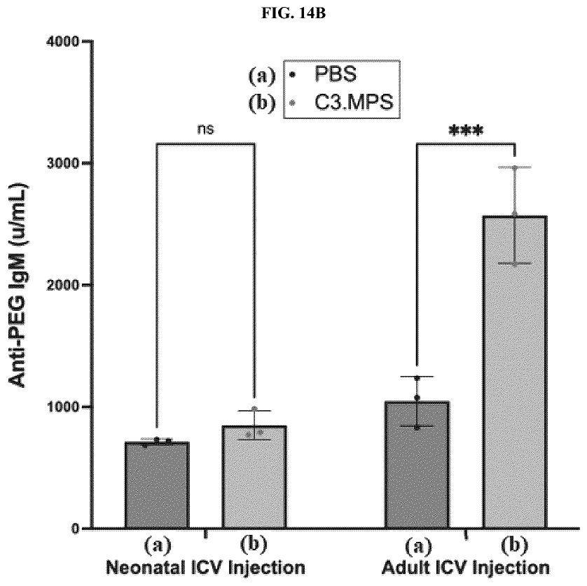

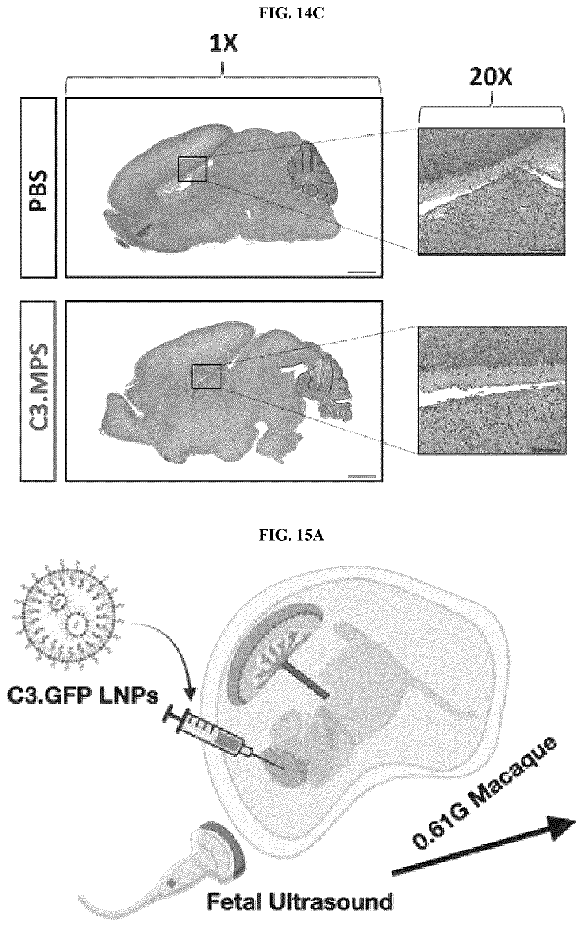

To extend the characterization of the safety of C3.MPS LNPs described herein, the acute, systemic immune response following LNP treatment was examined. A separate cohort of Idua-W392X mice was injected ICV at P0 with C3.MPS LNPs at a dose of 1 mg/kg and then bled at 24 hours prior to serum cytokine analysis. Serum from PBS-injected mice and mice injected with LNPs formulated with DLin-MC3-DMA ionizable lipid (MC3.MPS) instead of C₃ ionizable lipid were utilized as controls. Out of an array of 25 cytokines, 4 were significantly elevated in C3.MPS LNP-treated neonates compared to PBS-treated controls (FIG. 14A), including G-CSF (p<0.05), IL-la (p<0.05), Ip10 (p<0.05), and MCP-1 (p<0.05). However, there was no significant difference between the cytokine levels of G-CSF, IL-la, and MCP-1 in C3.MPS LNP-treated neonates and MC3.MPS LNP-treated neonates, while Ip10 was significantly increased in the latter group (p<0.0001). MC3.MPS LNPs also elicited an additional cytokine response, producing elevated levels of IL-9 (p<0.0001) and GM-CSF (p<0.0001). Taken together, these data suggest that while C3.MPS LNPs produce an acute serum cytokine response, this reaction is at a lower level than that produced by FDA-approved MC3 LNPs.

Recent studies have revealed that PEGylated therapies give rise to anti-PEG antibodies in both animal models and in patients. While PEGylation of LNPs attracts a water shell, reducing adsorption of opsonins and evading recognition of LNPs by the mononuclear phagocyte system, antibodies to PEG-lipid may cause adverse immune effects, reduce therapeutic efficacy, and limit the potential for repeat dosing due to the accelerated blood clearance phenomenon. To characterize the anti-PEG antibody response elicited by C3.MPS LNPs, scrum was analyzed from a separate cohort of Idua-W392X mice injected ICV at P0 with C3.MPS LNPs at a dose of 1 mg/kg and then terminally bled one week later. No significant difference in the serum anti-PEG IgM level was observed between C3.MPS LNP-treated and PBS-treated neonates

Interestingly, when the same experiment was repeated in adult Idua-W392X mice, a 2.5-fold increase in serum anti-PEG IgM antibodies in C3.MPS LNP-treated mice compared to PBS-treated mice (p<0.05) was observed (FIG. 14B). These data are consistent with previous results demonstrating that the immune system of a P0 mouse is immature, similar to that of a developing human fetus, and imply that a mature immune system, as is present in an adult mouse, is capable of producing an anti-PEG IgM response to C3.MPS LNPs.

### Example 7: C3 LNPs Enable mRNA Delivery to Cerebral Ventricular Cells in a Fetal Macaque

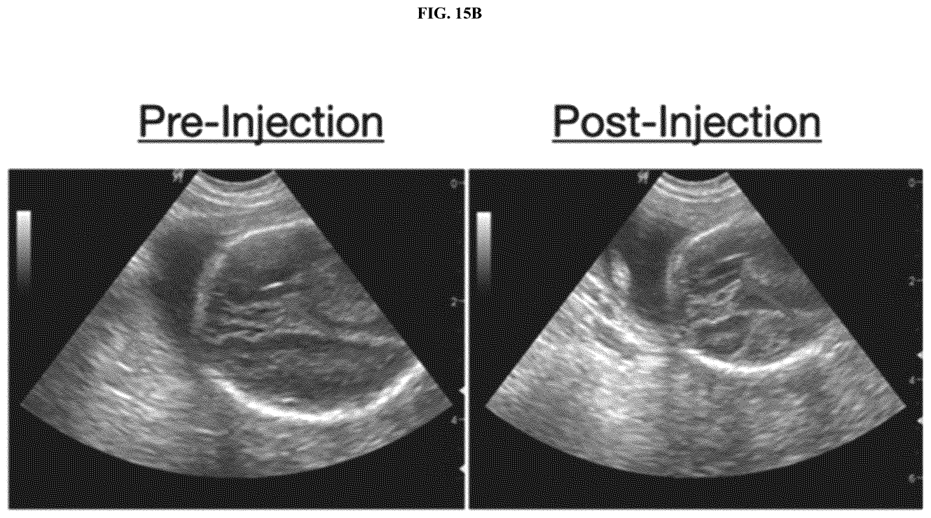

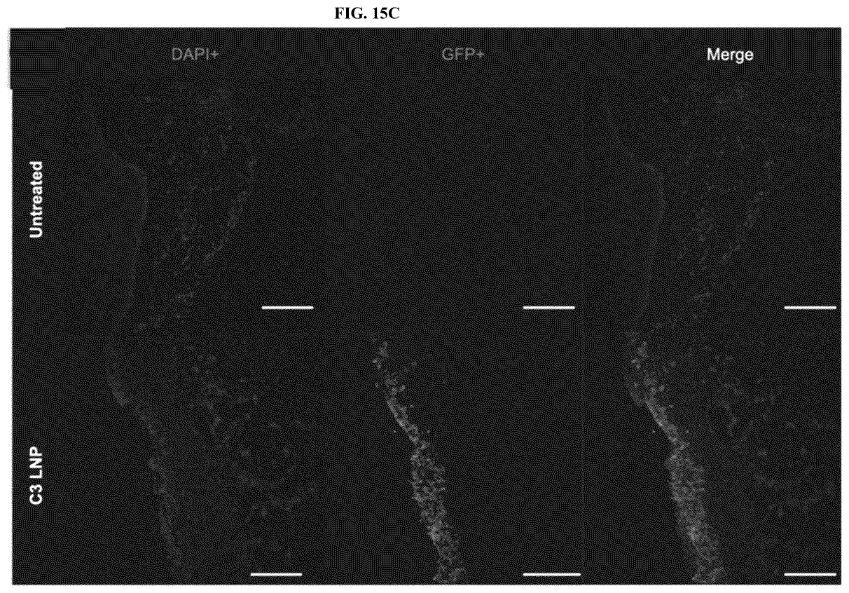

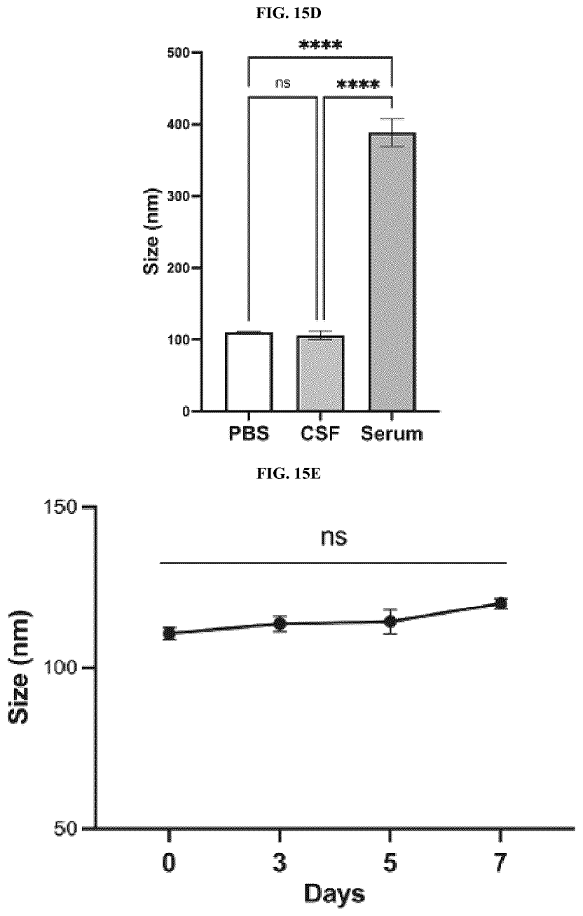

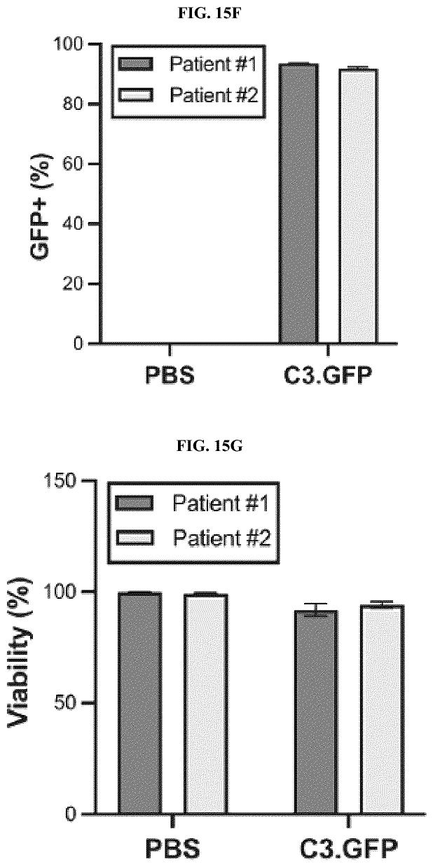

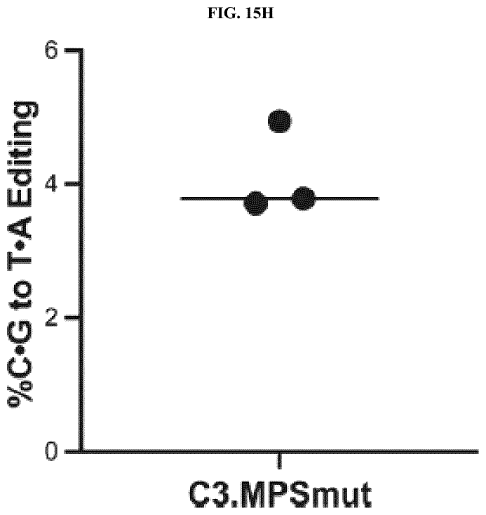

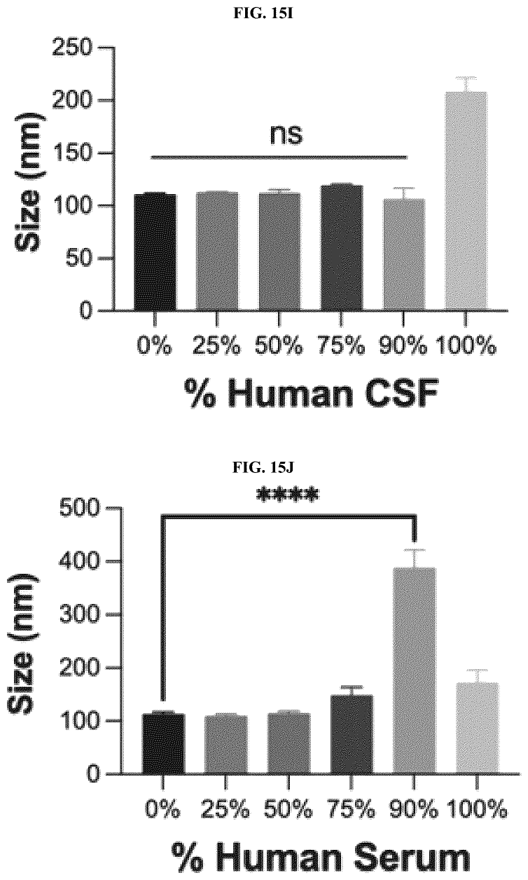

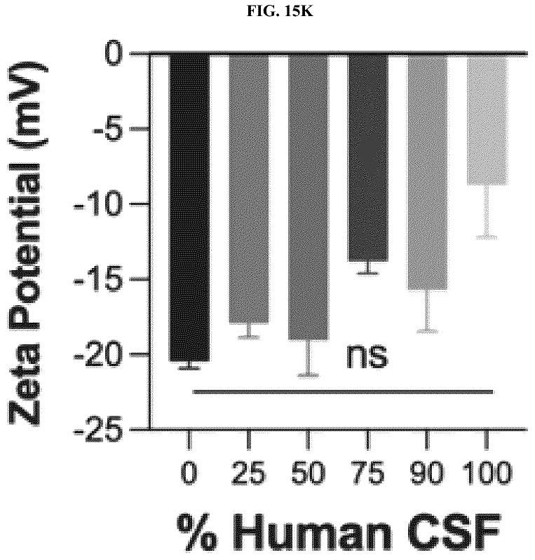

To demonstrate the translational potential of C3 LNPs, demonstration LNP-mediated mRNA delivery to the fetal brain in a nonhuman primate model was sought. Using transabdominal ultrasound, the lateral cerebral ventricles of a 0.61G Macaca fascicularis, which approximates a mid-gestation human fetus, were identified and C3 LNPs encapsulating GFP mRNA (C₃.GFP) were injected ICV at a dose of 1 mg/kg (FIG. 15A). Successful delivery was confirmed by observation of transient ventricular swelling (FIG. 15B). Forty-eight hours after injection, the fetus was delivered by caesarian section and GFP expression in the brain was assessed by immunohistochemistry. Similar to the R26^{mT/mG} mouse studies, GFP expression was observed in the cells lining the lateral ventricles (FIG. 15C). No discrete GFP+ cells were observed in the brain of an untreated control animal. This proof-of-principle experiment demonstrates that C3 LNPs can deliver mRNA to ventricular cells in the fetal brain of a nonhuman primate, while highlighting the need for future optimization to enhance targeting of other regions of the brain.

### Example 8: LNPs demonstrate translational potential in patient-derived brain tissues

To further evaluate the potential clinical relevance of C3 LNPs for mRNA delivery to the brain, their performance was tested in three distinct patient-derived samples. An assay was previously developed to evaluate the stability of LNPs in fetal fluids and demonstrated that LNPs that are more stable ex vivo had increased mRNA delivery in utero. To characterize the stability of C3 LNPs within the fluid in which they would ultimately be delivered, C3.MPS LNPs were incubated in cerebrospinal fluid (CSF) derived from a pediatric patient and assessed for stability via size and charge measurements (FIGS. 15D-15E and FIGS. 15I-15K). No significant change in size or PDI at all concentrations of LNPs within the CSF were observed. In contrast, incubation of C3.MPS LNPs in human serum resulted in significant aggregation and clustering. Previous studies have reported that zeta potential measurements of LNPs become more negative at increasing concentrations of protein-rich fluid. However, the zeta potential of C3.MPS LNPs did not change at increasing concentrations of human CSF, implying that C3 LNPs avoid significant protein adhesion that can reduce cellular uptake.

Next, primary brain cells were cultured from cerebral cortex tissue of two pediatric patients undergoing neurosurgical procedures (FIG. 15E). C3.GFP LNPs were applied to these primary cells, resulting in >90% transfection (FIG. 15F) with >90% viability (FIG. 15G) in both patient samples after 24 hours. Next, demonstration of C3 LNP-mediated base editing in human brain tissue was sought. Using an established protocol, precision cut slices of pediatric brain tissue were generated and cultured ex vivo in a 50:50 mixture of human CSF and media. C3 LNPs encapsulating a cytosine base editor and an sgRNA to introduce a T→C mutation at the W402X locus (C3.MPSmut LNPs), the most common mutation in MPS-IH patients, were formulated and used to base edit this ex vivo tissue. Next generation sequencing showed that 6% of cells carried the mutation introduced by C3.MPSmut LNPs (FIG. 15H, p<0.01). Thus, in this ex vivo human CNS tissue model, C3 LNPs successfully mediated co-delivery of a functional base editor and sgRNA.

Sequence Listing

SEQ ID NO: 1
ACTCTAGGCAGAGGTCTCAAAGG
SEQ ID NO: 2
CCAGAGCTGCTCCTCATCTGCGG
SEQ ID NO: 3
TGCTAGGTATGAGAGAGCCA
SEQ ID NO: 4
AGTGTAGATGAGGACTGTGGT
SEQ ID NO: 5
CAATGCCTTCCTGAGCTACCAC
SEQ ID NO: 6
AGGTAGCGCGTGACGTAGAC
SEQ ID NO: 7
GTTCTAGACTGAGGTCTCAAGGG
SEQ ID NO: 8
ACTCTAGGCTAGAGTCTCAAAGG
SEQ ID NO: 9
ACTTTTGACAGAGGTATCAAGGG
SEQ ID NO: 10
ATTCCAGCCAGAGGTATCAAAGG
SEQ ID NO: 11
AGTTCAGACAGAGGTCTCAAAGG
SEQ ID NO: 12
GCTCCAGGCAGAGGTCCCAGGGG
SEQ ID NO: 13
GAACTAAGCAGAGGTCTCAAAGG
SEQ ID NO: 14
GCTCTGAGCAGAGGTCCCAACGG
SEQ ID NO: 15
ACTCTACACAGAGGTACCAATGG

### Enumerated Embodiments

The following exemplary embodiments are provided, the numbering of which is not to be construed as designating levels of importance:

Embodiment 1 provides a lipid nanoparticle (LNP) composition comprising:
(a) at least one ionizable lipid;
(b) at least one helper lipid;
(c) cholesterol;
(d) at least one conjugated lipid;
(e) nucleic acid cargo comprising at least one mRNA and at least one sgRNA, wherein the nucleic acid cargo are at least partially encapsulated in the LNP.

Embodiment 2 provides the LNP of Embodiment 1, wherein the at least one ionizable lipid comprises an ionizable lipid of Formula (I), or a salt, solvate, stereoisomer, or isotopologue thereof:

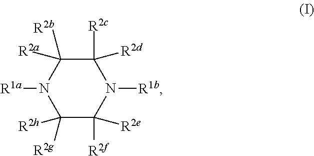

wherein:
R^{1a} and R^{1b} are each independently

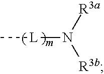

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴) (R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴) (R⁵), —C(═O)R⁴, and R⁴,
wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of (optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

Embodiment 3 provides the LNP of Embodiment 2, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R²⁶, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2g}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 4 provides the LNP of Embodiment 3, wherein each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR⁵)CH₂—, —(CH₂)₂NR^{3c}—,

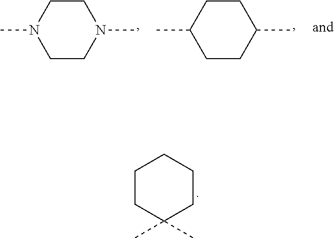

Embodiment 5 provides the LNP of any one of Embodiments 2-4, wherein the ionizable lipid of Formula (I) is selected from the group consisting of:

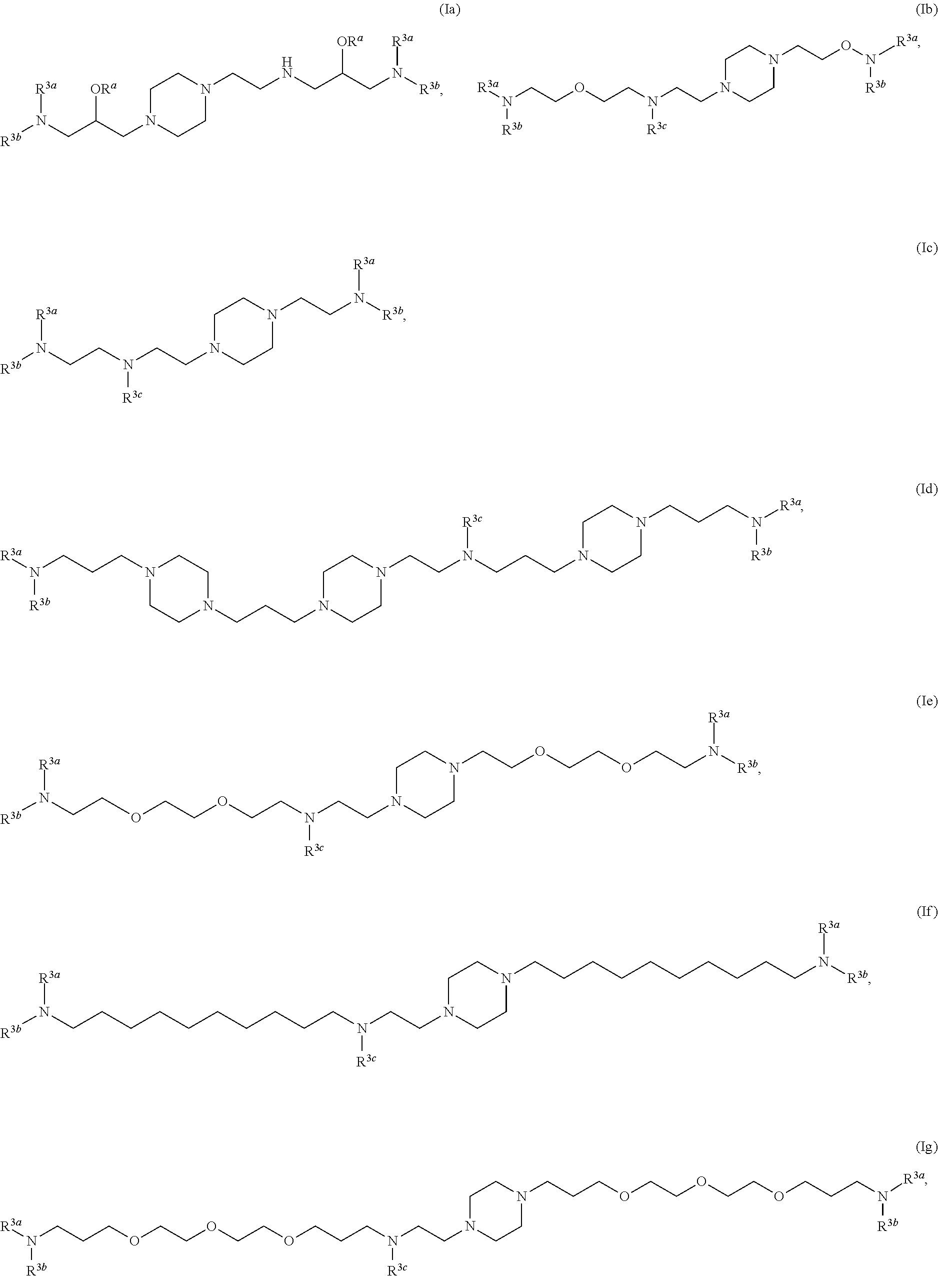

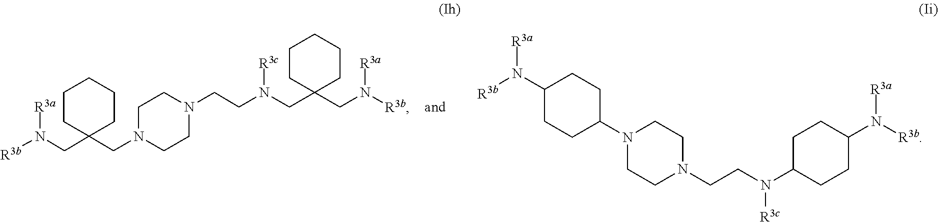

Embodiment 6 provides the LNP of any one of Embodiments 2-5, wherein each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O) NH (optionally substituted C₁-C₂₈ alkyl).

Embodiment 7 provides the LNP of any one of Embodiments 2-6, wherein each occurrence of R³⁸, R^{3b}, and R^{3c} is independently selected from the group consisting of CH₂CH(OH) (CH₂)₉CH₃, —CH₂CH(OH)(CH₂)₁₁CH₃, and —CH₂CH(OH)(CH₂)₁₃CH₃.

Embodiment 8 provides the LNP of any one of Embodiments 2-7, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 9 provides the LNP of any one of Embodiments 2-8, wherein the ionizable lipid of Formula (I) is:

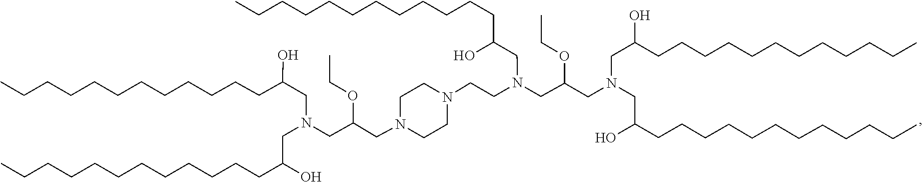 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]

### 1,1′-((3-(4-(2-((3-(bis(2-hydroxytetradecyl)amino)-2-ethoxypropyl) (2-hydroxytetradecyl)amino)ethyl) piperazin-1-yl)-2-ethoxypropyl) azanediyl)bis(tetradecan-2-ol) (C3)

Embodiment 10 provides the LNP of any one of Embodiments 1-9, wherein the at least one ionizable lipid comprises about 30 mol % to about 60 mol % of the LNP.

Embodiment 11 provides the LNP of any one of Embodiments 1-10, wherein the at least one ionizable lipid comprises about 35 mol % of the LNP.

Embodiment 12 provides the LNP of any one of Embodiments 1-11, wherein the helper lipid comprises at least one selected from the group consisting of dioleoylphosphatidylethanolamine (DOPE) and distearoylphosphatidylcholine (DSPC).

Embodiment 13 provides the LNP of any one of Embodiments 1-12, wherein the at least one helper lipid comprises about 1 to about 25 mol % of the LNP.

Embodiment 14 provides the LNP of any one of Embodiments 1-13, wherein the at least one helper lipid comprises about 16 mol % of the LNP.

Embodiment 15 provides the LNP of Embodiment 14, wherein the helper lipid is DOPE.

Embodiment 16 provides the LNP of any one of Embodiments 1-15, wherein the cholesterol comprises about 20 to about 60 mol % of the LNP.

Embodiment 17 provides the LNP of any one of Embodiments 1-16, wherein cholesterol comprises about 46.5 mol % of the LNP.

Embodiment 18 provides the LNP of any one of Embodiments 1-17, wherein the at least one conjugated lipid comprises about 0.1 to about 10.0 mol % of the LNP.

Embodiment 19 provides the LNP of any one of Embodiments 1-18, wherein the at least one conjugated lipid comprises about 2.5 mol % of the LNP.

Embodiment 20 provides the LNP of any one of Embodiments 1-19, wherein the at least one conjugated lipid comprises C14-PEG.

Embodiment 21 provides the LNP of any one of Embodiments 1-20, wherein the LNP has a molar ratio of (a):(b):(c):(d) of about 35:16:46.5:2.5.

Embodiment 22 provides the LNP of any one of Embodiments 1-21, wherein the LNP has a ratio of mRNA to sgRNA ranging from about 10:1 to about 0.1:1 (mRNA:sgRNA).

Embodiment 23 provides the LNP of any one of Embodiments 1-22, wherein the LNP has a ratio of mRNA to sgRNA of about 3:1 (mRNA:sgRNA).

Embodiment 24 provides the LNP of any one of Embodiments 1-23, wherein the LNP has a ratio of ionizable lipid to total nucleic acid (ionizable lipid:mRNA+sgRNA) ranging from about 5:1 to about 30:1.

Embodiment 25 provides the LNP of any one of Embodiments 1-24, wherein the LNP has a ratio of ionizable lipid to total nucleic acid (ionizable lipid:mRNA+sgRNA) of about 10:1.

Embodiment 26 provides the LNP of any one of Embodiments 1-25, wherein the mRNA encodes a base editor.

Embodiment 27 provides the LNP of Embodiment 26, wherein the base editor is selected from the group consisting of a cytosine base editor and an adenine base editor.

Embodiment 28 provides the LNP of any one of Embodiments 1-27, wherein the nucleic acid cargo is selectively delivered to the brain of a subject.

Embodiment 29 provides a pharmaceutical composition comprising the LNP of any one of Embodiments 1-28 and a pharmaceutically acceptable carrier.

Embodiment 30 provides a method of delivering a cargo to the brain of a subject in need thereof, the method comprising administering to the subject a therapeutically effective amount of at least one lipid nanoparticle (LNP) comprising:
(a) at least one ionizable lipid;
(b) at least one helper lipid;
(c) cholesterol;
(d) at least one conjugated lipid;
(e) at least one cargo molecule, wherein the at least one cargo molecule is at least partially encapsulated in the LNP.

Embodiment 31 provides the method of Embodiment 30, wherein the cargo is at least one selected from the group consisting of a nucleic acid molecule, small molecule, protein, therapeutic agent, antibody, and any combinations thereof.

Embodiment 32 provides the method of Embodiment 31, wherein the cargo is a nucleic acid molecule.

Embodiment 33 provides the method of Embodiment 31 or 32, wherein the nucleic acid molecule is a DNA molecule or a RNA molecule.

Embodiment 34 provides the method of any one of Embodiments 31-33, wherein the nucleic acid molecule is selected from the group consisting of mRNA, cDNA, pDNA, microRNA, siRNA, modified RNA, antagomir, antisense molecule, targeted nucleic acid, and any combination thereof.

Embodiment 35 provides a method of genome editing a mutated gene sequence associated with a disease or disorder in a subject in need thereof, the method comprising administering to the subject a therapeutically effective amount of the LNP of any one of Embodiments 1-28 or the pharmaceutical composition of Embodiment 29.

Embodiment 36 provides the method of Embodiment 35, wherein the disease or disorder is a neurological disease or disorder.

Embodiment 37 provides the method of Embodiment 36, wherein the neurological disease or disorder is at least one selected from the group consisting of a lysosomal storage disease, mitochondrial disease, Friedrich's ataxia, Huntington's disease.

Embodiment 38 provides the method of Embodiment 37, wherein the lysosomal storage disease is at least one selected from the group consisting of Farber disease, Krabbe disease, Fabry disease, Schindler disease, Sandhoff disease, Tay-Sachs disease, Gaucher disease, Niemann-Pick disease, Hurler syndrome, Scheie syndrome, Hurler-Scheie syndrome, Sanfilippo syndrome, Maroteaux-Lamy syndrome, Sly syndrome, hyaluronidase deficiency, sialidosis, I-cell disease, Santavuori-Haltia disease, Jansky-Bielschowsky disease, Batten-Spielmeyer-Vogt disease, Kufs disease, and Wolman disease.

Embodiment 39 provides the method of Embodiment 38, wherein the lysosomal storage disease is Hurler-Scheie syndrome.

Embodiment 40 provides the method of Embodiment 39, wherein the mutated gene comprises a G→A mutation in the Idua gene.

Embodiment 41 provides a method of treating, ameliorating, and/or preventing a lysosomal storage disease in a subject in need thereof, the method comprising administering to the subject a therapeutically effective amount of the lipid nanoparticle (LNP) of any one of Embodiments 1-28 or the pharmaceutical composition of Embodiment 29.

Embodiment 42 provides the method of Embodiment 41, wherein the lysosomal storage disease is at least one selected from the group consisting of Farber disease, Krabbe disease, Fabry disease, Schindler disease, Sandhoff disease, Tay-Sachs disease, Gaucher disease, Niemann-Pick disease, Hurler syndrome, Scheie syndrome, Hurler-Scheie syndrome, Sanfilippo syndrome, Maroteaux-Lamy syndrome, Sly syndrome, hyaluronidase deficiency, sialidosis, I-cell disease, Santavuori-Haltia disease, Jansky-Bielschowsky disease, Batten-Spielmeyer-Vogt disease, Kufs disease, and Wolman disease.

Embodiment 43 provides the method of Embodiment 42, wherein the lysosomal storage disease is Hurler-Scheie syndrome.

Embodiment 44 provides the method of Embodiment 43, wherein the mutated gene comprises a G→A mutation in the Idua gene.

Embodiment 45 provides the method of any one of Embodiments 35-44, wherein the at least one ionizable lipid comprises an ionizable lipid of Formula (I), or a salt, solvate, stereoisomer, or isotopologue thereof:

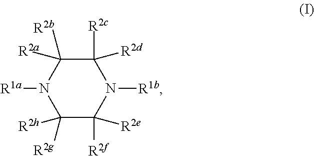

wherein:
R^{1a} and R^{1b} are each independently

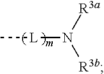

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴) (R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴) (R⁵), —C(—O)R⁴, and R⁴,
wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of (optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

Embodiment 46 provides the method of Embodiment 45, wherein at least one of the following applies:
(a) at least one selected from the group consisting of R^{2a}, R²⁶, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R²⁶, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R²⁶, R^{2c}, R^{2d}, R^{2g}, R^{2f}, R^{2g}, and R^{2h} are H;
(f) at least six selected from the group consisting of R^{2a}, R²⁶, R^{2c}, R^{2d}, R^{2e}, R^{2f}, R^{2g}, and R^{2h} are H;
(g) at least seven selected from the group consisting of R^{2a}, R²⁶, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 47 provides the method of Embodiment 45 or 46, wherein each occurrence of L is independently selected from the group consisting of —CH₂—, —(CH₂)₂—, —(CH₂)₃—, —(CH₂)₁₀—, (CH₂)₂O—, —(CH₂)₃O—, —CH₂CH(OR⁵)CH₂—, —(CH₂)₂NR^{3c}—,

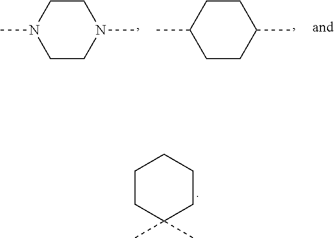

Embodiment 48 provides the method of any one of Embodiments 45-47, wherein the ionizable lipid of Formula (I) is selected from the group consisting of:

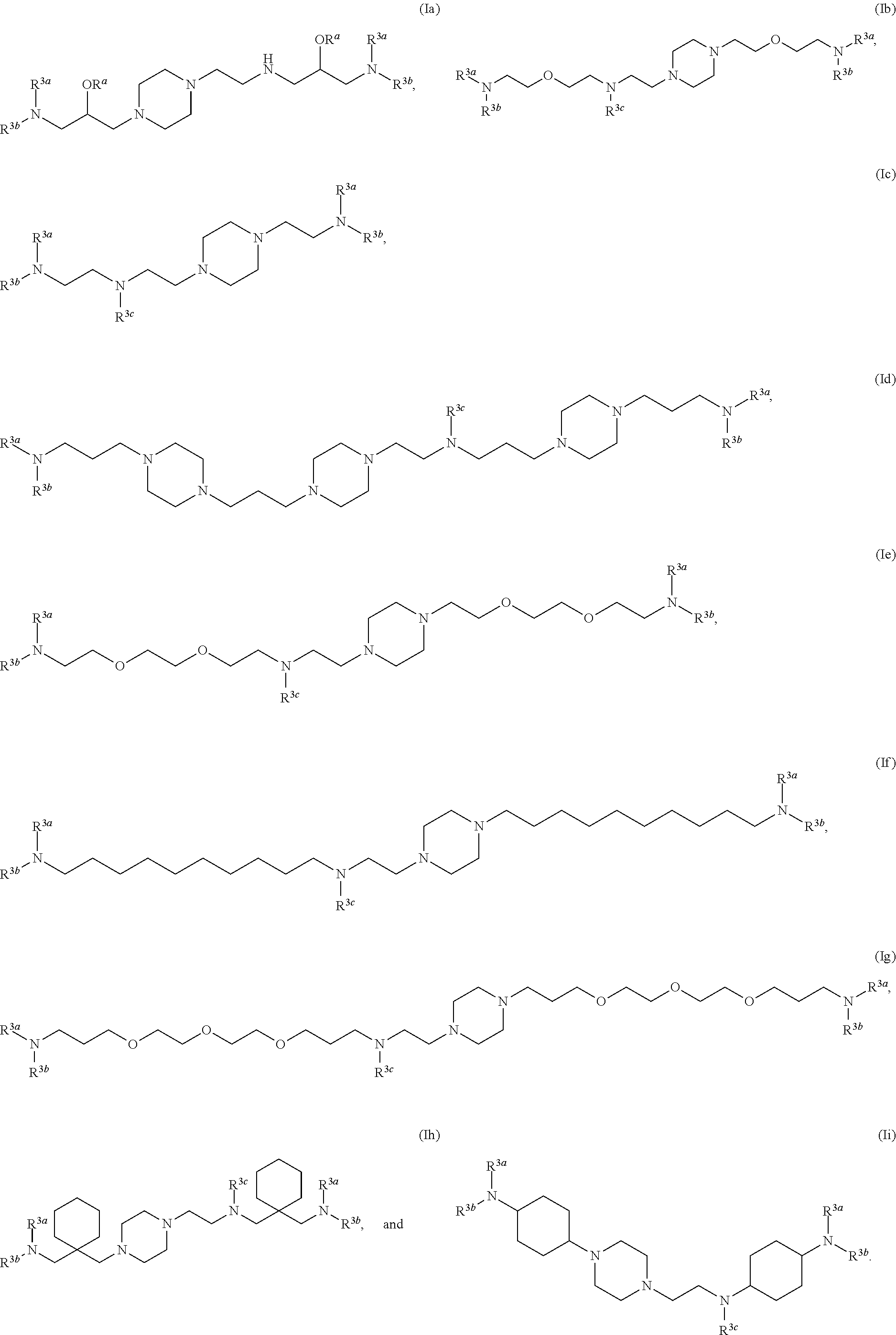

Embodiment 49 provides the method of any one of Embodiments 45-48, wherein each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O) NH (optionally substituted C₁-C₂₈ alkyl).

Embodiment 50 provides the method of any one of Embodiments 45-49, wherein each occurrence of R^{3a}, R^{3b}, and R^{3c} is independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 51 provides the method of any one of Embodiments 45-50, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 52 provides the method of any one of Embodiments 45-51, wherein the ionizable lipid of Formula (I) is:

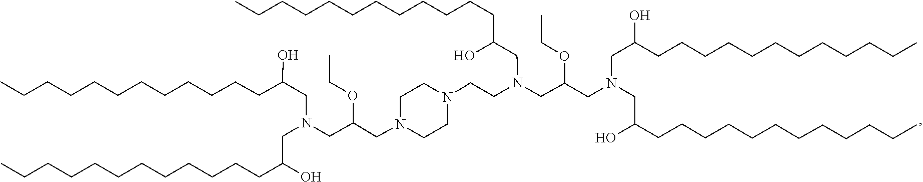 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]

### 1,1′-((3-(4-(2-((3-(bis(2-hydroxytetradecyl)amino)-2-ethoxypropyl) (2-hydroxytetradecyl)amino)ethyl) piperazin-1-yl)-2-ethoxypropyl) azanediyl)bis(tetradecan-2-ol) (C3)

Embodiment 53 provides the method of any one of Embodiments 35-52, wherein the at least one ionizable lipid comprises about 30 mol % to about 60 mol % of the LNP.

Embodiment 54 provides the method of any one of Embodiments 35-53, wherein the at least one ionizable lipid comprises about 35 mol % of the LNP.

Embodiment 55 provides the method of any one of Embodiments 35-54, wherein the helper lipid comprises at least one selected from the group consisting of diolcoylphosphatidylethanolamine (DOPE) and distearoylphosphatidylcholine (DSPC).

Embodiment 56 provides the method of any one of Embodiments 35-55, wherein the at least one helper lipid comprises about 1 to about 25 mol % of the LNP.

Embodiment 57 provides the method of any one of Embodiments 35-56, wherein the at least one helper lipid comprises about 16 mol % of the LNP.

Embodiment 58 provides the method of Embodiment 57, wherein the helper lipid is DOPE.

Embodiment 59 provides the method of any one of Embodiments 35-58, wherein the cholesterol comprises about 20 to about 60 mol % of the LNP.

Embodiment 60 provides the method of any one of Embodiments 35-59, wherein cholesterol comprises about 46.5 mol % of the LNP.

Embodiment 61 provides the method of any one of Embodiments 35-60, wherein the at least one conjugated lipid comprises about 0.1 to about 10.0 mol % of the LNP.

Embodiment 62 provides the method of any one of Embodiments 35-61, wherein the at least one conjugated lipid comprises about 2.5 mol % of the LNP.

Embodiment 63 provides the method of any one of Embodiments 35-62, wherein the at least one conjugated lipid comprises C14-PEG.

Embodiment 64 provides the method of any one of Embodiments 35-63, wherein the LNP has a molar ratio of (a):(b):(c):(d) of about 35:16:46.5:2.5.

Embodiment 65 provides the method of any one of Embodiments 35-64, wherein the LNP has a ratio of mRNA to sgRNA ranging from about 10:1 to about 0.1:1 (mRNA:sgRNA).

Embodiment 66 provides the method of any one of Embodiments 35-65, wherein the LNP has a ratio of mRNA to sgRNA of about 3:1 (mRNA:sgRNA).

Embodiment 67 provides the method of any one of Embodiments 35-66, wherein the LNP has a ratio of ionizable lipid to total nucleic acid (ionizable lipid:mRNA+sgRNA) ranging from about 5:1 to about 30:1.

Embodiment 68 provides the method of any one of Embodiments 35-67, wherein the LNP has a ratio of ionizable lipid to total nucleic acid (ionizable lipid:mRNA+sgRNA) of about 10:1.

Embodiment 69 provides the method of any one of Embodiments 35-68, wherein the mRNA encodes a base editor.

Embodiment 70 provides the method of Embodiment 69, wherein the base editor is selected from the group consisting of a cytosine base editor and an adenine base editor.

Embodiment 71 provides the method of any one of Embodiments 35-70, wherein the nucleic acid cargo is selectively delivered to the brain of a subject.

Embodiment 72 provides the method of any one of Embodiments 35-71, wherein the administration is intracerebrovenctricular (ICV).

Embodiment 73 provides the method of any one of Embodiments 35-72, wherein the administration is in utero.

Embodiment 74 provides the method of any one of Embodiments 35-73, wherein the subject is a mammal.

Embodiment 75 provides the method of Embodiment 74, wherein the mammal is in an embryonic, fetal, perinatal, or neonatal stage of development.

Embodiment 76 provides the method of Embodiment 74 or 75, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A lipid nanoparticle (LNP) composition comprising:
(a) at least one ionizable lipid;
(b) at least one helper lipid;
(c) cholesterol;
(d) at least one conjugated lipid;
(e) a nucleic acid cargo comprising at least one mRNA and at least one sgRNA, wherein the nucleic acid cargo are at least partially encapsulated in the LNP.

2. The LNP of claim 1, wherein the at least one ionizable lipid comprises an ionizable lipid of Formula (I), or a salt, solvate, stereoisomer, or isotopologue thereof: 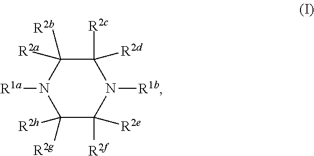
wherein:
R^{1a} and R^{1b} are each independently 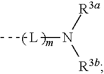
R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴) (R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴) (R⁵), —C(═O)R⁴, and R⁴,
wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of (optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently 1, 2, 3, or 4.

3. The LNP of claim 2, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H;
(h) each of R^{2a}, R^{2b}, R^{2e}, R^{2d}, R^{2e}, R^{2f}, R^{2g}, and R^{2h} are H;
(i) each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR⁵)CH₂—, —(CH₂)₂NR^{3c}—, 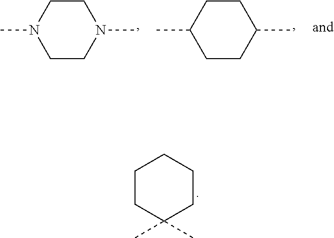
(j) each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O) NH (optionally substituted C₁-C₂₈ alkyl), optionally wherein each occurrence of R^{3a}, R^{3b}, and R^{3c} is independently selected from the group consisting of —CH₂CH(OH)(CH₂)₉CH₃, —CH₂CH(OH)(CH₂)₁₁CH₃, and —CH₂CH(OH)(CH₂)₁₃CH₃; and
(k) each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(—O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

4. The LNP of claim 2, wherein the ionizable lipid of Formula (I) is selected from the group consisting of:

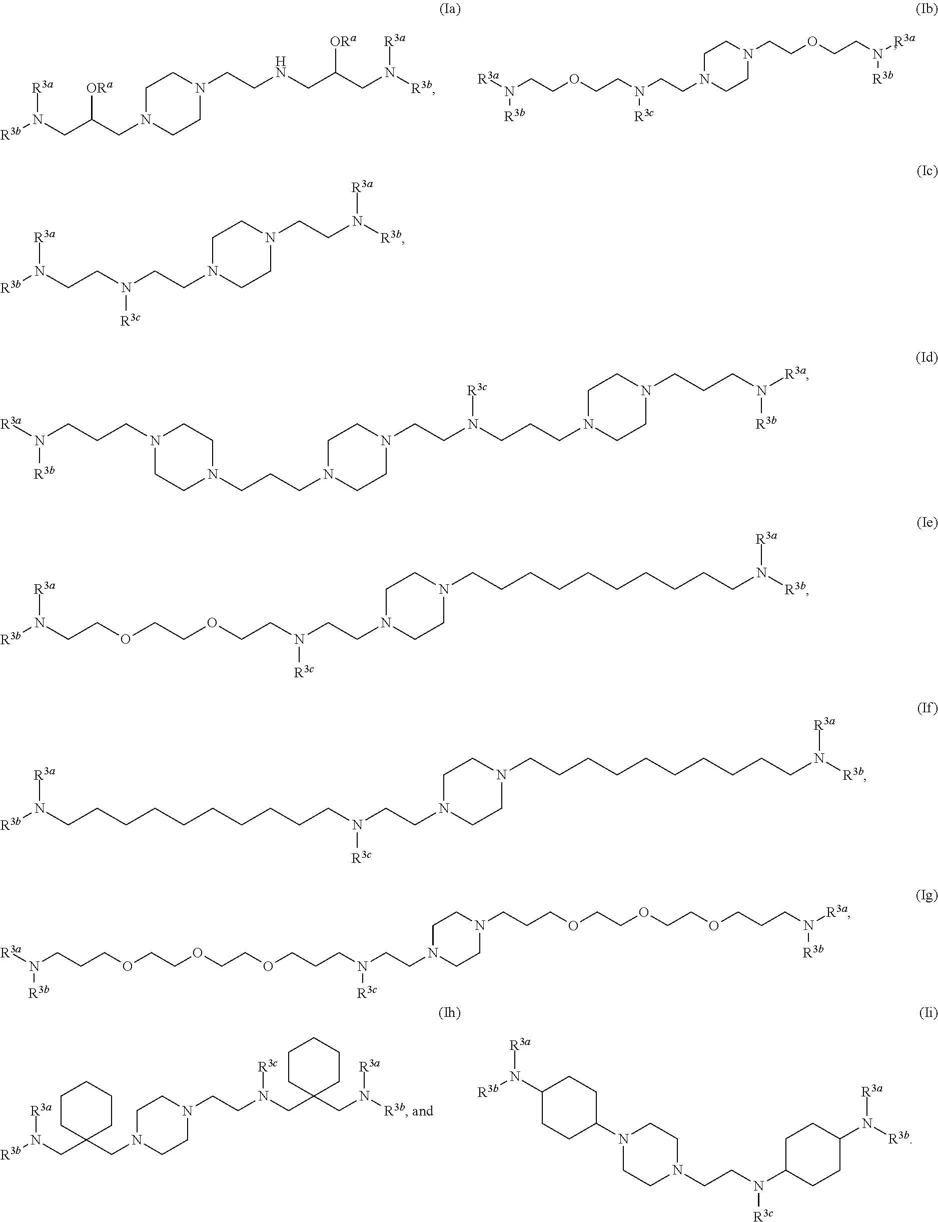

5. The LNP of claim 1, wherein the at least one of the following applies:
(a) the ionizable lipid comprises about 30 mole to about 60 mole of the LNP, optionally wherein the at least one ionizable lipid comprises about 35 mol % of the LNP;
(b) the at least one helper lipid comprises about 1 to about 25 mol % of the LNP, optionally wherein the at least one helper lipid comprises about 16 mol % of the LNP;
(c) the cholesterol comprises about 20 to about 60 mol % of the LNP, optionally wherein cholesterol comprises about 46.5 mol % of the LNP; and
(d) the at least one conjugated lipid comprises about 0.1 to about 10.0 mol % of the LNP, optionally wherein the at least one conjugated lipid comprises about 2.5 mol % of the LNP.

6. The LNP of claim 1, wherein at least one of the following applies:
(a) the ionizable lipid is: 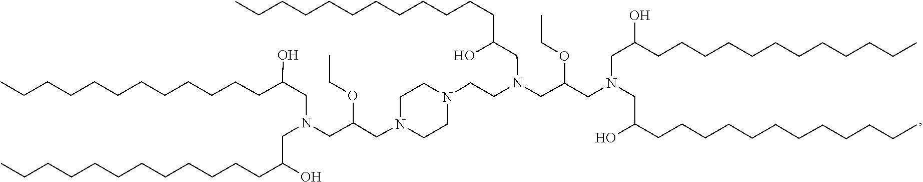 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]
1,1′-((3-(4-(2-((3-(bis(2-hydroxytetradecyl)amino)-2-ethoxypropyl) (2-hydroxytetradecyl)amino)ethyl) piperazin-1-yl)-2-ethoxypropyl) azanediyl)bis(tetradecan-2-ol) (C3);
(b) the helper lipid comprises at least one selected from the group consisting of dioleoylphosphatidylethanolamine (DOPE) and distearoylphosphatidylcholine (DSPC);
(c) the helper lipid is DOPE; and
(d) the at least one conjugated lipid comprises C14-PEG.

7. The LNP of claim 1, wherein the LNP has a molar ratio of (a):(b):(c):(d) of about 35:16:46.5:2.5.

8. The LNP of claim 1, wherein at least one of the following applies:
(a) the LNP has a ratio of mRNA to sgRNA ranging from about 10:1 to about 0.1:1 (mRNA:sgRNA ), optionally wherein the LNP has a ratio of mRNA to sgRNA of about 3:1 (mRNA:sgRNA);
(b) the LNP has a ratio of ionizable lipid to total nucleic acid (ionizable lipid:mRNA+sgRNA) ranging from about 5:1 to about 30:1, optionally wherein the LNP has a ratio of ionizable lipid to total nucleic acid (ionizable lipid:mRNA+sgRNA) of about 10:1.

9. The LNP of claim 1, wherein the mRNA encodes a base editor, optionally wherein the base editor is selected from the group consisting of a cytosine base editor and an adenine base editor.

10. The LNP of claim 1, wherein the nucleic acid cargo is selectively delivered to the brain of a subject.

11. A pharmaceutical composition comprising the LNP of claim 1 and a pharmaceutically acceptable carrier.

12. A method of delivering a cargo to the brain of a subject in need thereof, the method comprising administering to the subject a therapeutically effective amount of at least one lipid nanoparticle (LNP) comprising:
(a) at least one ionizable lipid of claim 2;
(b) at least one helper lipid;
(c) cholesterol;
(d) at least one conjugated lipid;
(e) at least one cargo molecule, wherein the at least one cargo molecule is at least partially encapsulated in the LNP.

13. The method of claim 12, wherein the cargo is at least one selected from the group consisting of a nucleic acid molecule, small molecule, protein, therapeutic agent, antibody, and any combinations thereof, optionally wherein the nucleic acid molecule is a DNA molecule or a RNA molecule, and optionally wherein the DNA molecule or a RNA molecule is selected from the group consisting of mRNA, cDNA, pDNA, microRNA, siRNA, modified RNA, antagomir, antisense molecule, targeted nucleic acid, and any combination thereof.

14. The method of claim 12, wherein at least one of the following applies:
(a) the administration is intracerebroventricular (ICV);
(b) the administration is in utero; and
(c) the subject is a mammal, optionally wherein the mammal is in an embryonic, fetal, perinatal, or neonatal stage of development, and optionally wherein the mammal is a human

15. A method of genome editing a mutated gene sequence associated with a disease or disorder in a subject in need thereof, the method comprising administering to the subject a therapeutically effective amount of the LNP of claim 1 or a pharmaceutical composition thereof.

16. The method of claim 15, wherein at least one of the following applies:
(a) the disease or disorder is a neurological disease or disorder, optionally wherein the neurological disease or disorder is at least one selected from the group consisting of a lysosomal storage disease, mitochondrial disease, Friedrich's ataxia, Huntington's disease, and optionally wherein the lysosomal storage disease is at least one selected from the group consisting of Farber disease, Krabbe disease, Fabry disease, Schindler disease, Sandhoff disease, Tay-Sachs disease, Gaucher disease, Niemann-Pick disease, Hurler syndrome, Scheie syndrome, Hurler-Scheie syndrome, Sanfilippo syndrome, Maroteaux-Lamy syndrome, Sly syndrome, hyaluronidase deficiency, sialidosis, I-cell disease, Santavuori-Haltia disease, Jansky-Bielschowsky disease, Batten-Spielmeyer-Vogt disease, Kufs disease, and Wolman disease; and
(b) the mutated gene comprises a G→A mutation in the Idua gene.

17. The method of claim 15, wherein at least one of the following applies:
(a) the administration is intracerebroventricular (ICV);
(b) the administration is in utero; and
(c) the subject is a mammal, optionally wherein the mammal is in an embryonic, fetal, perinatal, or neonatal stage of development, and optionally wherein the mammal is a human.

18. A method of treating, ameliorating, and/or preventing a lysosomal storage disease in a subject in need thereof, the method comprising administering to the subject a therapeutically effective amount of the lipid nanoparticle (LNP) of claim 1 or a pharmaceutical composition thereof.

19. The method of claim 18, wherein at least one of the following applies:
(a) the lysosomal storage disease is at least one selected from the group consisting of Farber disease, Krabbe disease, Fabry disease, Schindler disease, Sandhoff disease, Tay-Sachs disease, Gaucher disease, Niemann-Pick disease, Hurler syndrome, Scheie syndrome, Hurler-Scheie syndrome, Sanfilippo syndrome, Maroteaux-Lamy syndrome, Sly syndrome, hyaluronidase deficiency, sialidosis, I-cell disease, Santavuori-Haltia disease, Jansky-Bielschowsky disease, Batten-Spielmeyer-Vogt disease, Kufs disease, and Wolman disease, optionally wherein the lysosomal storage disease is Hurler-Scheie syndrome; and
(b) the mutated gene comprises a G→A mutation in the Idua gene.

20. The method of claim 18, wherein at least one of the following applies:
(a) the administration is intracerebrovenctricular (ICV);
(b) the administration is in utero;
(c) the subject is a mammal, optionally wherein the mammal is in an embryonic, fetal, perinatal, or neonatal stage of development, and optionally wherein the mammal is a human.


SEQUENCE LISTING (15 sequences)

SEQ ID NO: 1 - DNA, 23 bp, synthetic construct
  source 1..23: mol_type = other DNA
actctaggcagaggtctcaaagg

SEQ ID NO: 2 - DNA, 23 bp, synthetic construct
  source 1..23: mol_type = other DNA
ccagagctgctcctcatctgcgg

SEQ ID NO: 3 - DNA, 20 bp, synthetic construct
  source 1..20: mol_type = other DNA
tgctaggtatgagagagcca

SEQ ID NO: 4 - DNA, 21 bp, synthetic construct
  source 1..21: mol_type = other DNA
agtgtagatgaggactgtggt

SEQ ID NO: 5 - DNA, 22 bp, synthetic construct
  source 1..22: mol_type = other DNA
caatgccttcctgagctaccac

SEQ ID NO: 6 - DNA, 20 bp, synthetic construct
  source 1..20: mol_type = other DNA
aggtagcgcgtgacgtagac

SEQ ID NO: 7 - DNA, 23 bp, synthetic construct
  source 1..23: mol_type = other DNA
gttctagactgaggtctcaaggg

SEQ ID NO: 8 - DNA, 23 bp, synthetic construct
  source 1..23: mol_type = other DNA
actctaggctagagtctcaaagg

SEQ ID NO: 9 - DNA, 23 bp, synthetic construct
  source 1..23: mol_type = other DNA
acttttgacagaggtatcaaggg

SEQ ID NO: 10 - DNA, 23 bp, synthetic construct
  source 1..23: mol_type = other DNA
attccagccagaggtatcaaagg

SEQ ID NO: 11 - DNA, 23 bp, synthetic construct
  source 1..23: mol_type = other DNA
agttcagacagaggtctcaaagg

SEQ ID NO: 12 - DNA, 23 bp, synthetic construct
  source 1..23: mol_type = other DNA
gctccaggcagaggtcccagggg

SEQ ID NO: 13 - DNA, 23 bp, synthetic construct
  source 1..23: mol_type = other DNA
gaactaagcagaggtctcaaagg

SEQ ID NO: 14 - DNA, 23 bp, synthetic construct
  source 1..23: mol_type = other DNA
gctctgagcagaggtcccaacgg

SEQ ID NO: 15 - DNA, 23 bp, synthetic construct
  source 1..23: mol_type = other DNA
actctacacagaggtaccaatgg